(19) United States
(12) Patent Application Publication
(10) Pub. No.: US 2025/0045925 A1
(43) Pub. Date: Feb. 6, 2025

(54) SEGMENTATION OF OPTICAL COHERENCE TOMOGRAPHY (OCT) IMAGES

(71) Applicant: Hoffmann-La Roche Inc., Little Falls, NJ (US)

(72) Inventors: Andreas MAUNZ, Basel (CH); Huanxiang LU, Préverenges (CH); Thomas Felix ALBRECHT, Basel (CH); Fethallah BENMANSOUR, Basel (CH); Yun Yvonna LI, Basel (CH)

(21) Appl. No.: 18/923,478

(22) Filed: Oct. 22, 2024

Related U.S. Application Data
(63) Continuation of application No. PCT/US2023/019644, filed on Apr. 24, 2023.
(60) Provisional application No. 63/333,995, filed on Apr. 22, 2022.

(51) Int. Cl. G06T 7/00; G06T 5/70; G06T 7/10; G16H 30/20; G16H 30/40

(52) U.S. Cl. CPC G06T 7/0012; G06T 5/70; G06T 7/10; G16H 30/20; G16H 30/40; G06T 2207/10101; G06T 2207/20084; G06T 2207/30041

(57) ABSTRACT

Systems and methods for performing automated retinal segmentation. Performing the automated retinal segmentation includes receiving an image input for a retina of a subject. Layer element data is generated using the image input and a first neural network. The layer element data identifying a set of retinal layer elements. Initial pathological element data is generated using the image input and a second neural network. The initial pathological element data identifies a set of retinal pathological elements. The initial pathological element data is refined using the layer element data to generate refined pathological element data. The refined pathological element data more accurately identifies the set of retinal pathological elements as compared to the initial pathological element data.

### CROSS-REFERENCE TO RELATED APPLICATIONS

This application is a continuation of International Application No. PCT/US2023/019644 filed on Apr. 24, 2023, and entitled “Segmentation of Optical Coherence Tomography (OCT) Images,” which claims priority to U.S. Provisional Application No. 63/333,995 filed on Apr. 22, 2022 and entitled “Segmentation of Optical Coherence Tomography (OCT) Images,” each of which is incorporated herein by reference in its entirety.

### FIELD

This application relates to retinal segmentation used in the diagnosis and/or treatment of ophthalmological diseases (or conditions), and more particularly, to automated retinal segmentation of optical coherence tomography (OCT) images using machine learning-based algorithms for the diagnosis and/or treatment of ophthalmological diseases (e.g., age-related macular degeneration (AMD), diabetic macular edema (DME), etc.).

### INTRODUCTION

Ophthalmologic diseases and conditions vary and can include retinal diseases and conditions. Retinal diseases may affect one or more parts of the retina, which is tissue at the back of the eye used to capture and convert light into signals (e.g., electrical, chemical) that are sent to the brain. Retinal diseases may lead to complications such as, for example, swelling of the macula (referred to as macular edema). Many retinal diseases affect vision and can lead to vision loss or, in some cases, blindness. Treatment may involve stopping or slowing disease to preserve, improve, or restore vision.

Age-related macular degeneration (AMD) is a leading cause of vision loss in subjects 50 years and older. AMD initially manifests as a dry type of AMD and can progress to a wet type of AMD. For the dry type, small deposits (drusen) form under the macula on the retina, causing the retina to deteriorate in time. For the wet type, which may also be referred to as neovascular AMD (nAMD), abnormal blood vessels originating in the choroid layer of the eye grow into the retina and leak fluid from the blood into the retina. Upon entering the retina, the fluid may distort the vision of a subject immediately, and over time, can damage the retina itself, for example, by causing the loss of photoreceptors in the retina. The fluid can cause the macula to separate from its base, resulting in severe and fast vision loss.

Diabetic macular edema (DME), a complication of diabetic retinopathy (DR), is oftentimes responsible for the vision loss experienced by patients living with diabetes. With DME, excess fluid accumulates in the extracellular space within the retina in the macular area (e.g., in the inner nuclear layer, outer plexiform layer, Henle's fiber layer, and subretinal space).

Optical coherence tomography (OCT) can provide a detailed scan of the macula to help detect macular degeneration, diabetic macular edema, and other ophthalmologic problems much earlier than was possible in the past.

To investigate the extent of the deterioration in a retina with, for example, AMD or DME, OCT images (e.g., time domain optical coherence tomography (TD-OCT) or spectral domain optical coherence tomography (SD-OCT) images) of the retina may be obtained and used for identifying features that may be associated with varying degenerative levels of disease (e.g., AMD, DME). SD-OCT is an imaging technique in which light is directed at the retina at various optical frequencies and in which the reflected light is collected to capture two-dimensional or three-dimensional, high-resolution, cross-sectional images of the retina via interferometric signals detected as a function of frequencies. Different features that are captured in the SD-OCT images can be identified via retinal segmentation and used in determining the severity of retinal disease, which may help guide the diagnosis and/or treatment of the disease. However, currently available techniques used in extracting, understanding, and/or interpreting such features may be plagued with tediousness and/or prone to error. Accordingly, the cumbersome nature of the retinal disease investigation process may be a limiting factor in the diagnosis and/or treatment of the disease. Thus, it may be desirable to have one or more methods and/or systems that recognize and take into account these issues.

### SUMMARY

In one or more embodiments, a method is provided for performing retinal segmentation. The method includes receiving an optical coherence tomography (OCT) image of a retina. A layer element image is generated using the OCT image and a first neural network, the layer element image identifying a set of retinal layer elements using a set of layer element indicators. An initial pathological element image is generated using the OCT image and a second neural network, the initial pathological element image visually identifying a set of retinal pathological elements using a set of pathological element indicators that assigns a different group of pixels to each retinal pathological element of the set of retinal pathological elements. The initial pathological element image is refined using the layer element image to generate a refined pathological element image. The refined pathological element image visually identifies the set of retinal pathological elements using the set of pathological element indicators, the set of pathological element indicators assigning an updated group of pixels to at least one retinal pathological element of the set of retinal pathological elements.

In one or more embodiments, a method is provided for performing retinal segmentation. The method includes receiving an optical coherence tomography (OCT) image of a retina and generating, via a neural network, a multi-channel map using the OCT image. The multi-channel map includes a plurality of segmented images in which each segmented image of the plurality of segmented images identifies a corresponding retinal layer of interest. A layer element image is generated using the multi-channel map, identifying a set of retinal layer elements using a set of layer element indicators. An initial pathological element image is refined using the layer element image to generate a refined pathological element image that visually identifies a set of retinal pathological elements using a set of pathological element indicators, wherein the refined pathological element image identifies at least one retinal pathological element in the set of retinal pathological elements more accurately than the initial pathological element image.

In one or more embodiments, a system for performing automated retinal segmentation is provided. The system comprises a non-transitory memory and a data processor coupled with the non-transitory memory. The data processor is configured to read instructions from the non-transitory memory to cause the system to perform operations comprising: receiving an optical coherence tomography (OCT) image of a retina; generating a layer element image using the OCT image and a first neural network, the layer element image identifying a set of retinal layer elements using a set of layer element indicators; gencrating an initial pathological element image using the OCT image and a second neural network, the initial pathological element image visually identifying a set of retinal pathological elements using a set of pathological element indicators that assigns a different group of pixels to each retinal pathological element of the set of retinal pathological elements; and refining the initial pathological element image using the layer element image to generate a refined pathological element image, the refined pathological element image visually identifying the set of retinal pathological elements using the set of pathological element indicators, the set of pathological element indicators assigning an updated group of pixels to at least one retinal pathological element of the set of retinal pathological elements.

In one or more embodiments, a method for performing automated retinal segmentation is provided. The method includes receiving an image input for a retina of a subject. Layer element data is generated using the image input and a first neural network. The layer element data identifying a set of retinal layer elements. Initial pathological element data is generated using the image input and a second neural network. The initial pathological element data identifies a set of retinal pathological elements. The initial pathological element data is refined using the layer element data to generate refined pathological element data. The refined pathological element data more accurately identifies the set of retinal pathological elements as compared to the initial pathological element data.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a retinal segmentation system, in accordance with various embodiments.

FIG. 2 illustrates an example process flow for performing retinal segmentation of optical coherence tomography (OCT) images using machine learning-based algorithms, in accordance with various embodiments.

FIG. 3 is a block diagram illustrating a neural network with a multi-channel learning method that can be used in a retinal segmentation system, in accordance with various embodiments.

FIG. 4 is a flowchart of a method of performing retinal segmentation, in accordance with various embodiments.

FIG. 5 is a flowchart of a method for generating a layer element image, in accordance with various embodiments.

FIG. 6 is a flowchart of a method for performing retinal segmentation, in accordance with various embodiments.

FIG. 7 is a flowchart of another method for performing automated retinal segmentation, in accordance with various embodiments.

FIGS. 8A and 8B are illustrations of retinal segmentation results in accordance with various embodiments.

FIG. 9 is a schematic diagram of an example neural network that can be used to implement a computer-based model in accordance with various embodiments.

FIG. 10 is a block diagram of a computer system in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

Various types of ophthalmological diseases (or conditions) may be detected, diagnosed, and/or treated using a detailed scan of the retina. As one example, neovascular age-related macular degeneration (nAMD) may be detected, diagnosed, and/or treated using a detailed scan of the retina in the macula region. As another example, diabetic macular edema (DME) may be detected, diagnosed, and/or treated using a detailed scan of the retina in the macular region. The embodiments described herein provide an improved technique for automated retinal segmentation of retinal images (e.g., retinal scans) that is more accurate and more reliable than existing methods for processing retinal images. More accurate and more reliable retinal segmentation may help ensure more accurate and thorough diagnostic and/or treatment solutions for patients with ophthalmological diseases such as, for example, but not limited to, nAMD and DME.

Retinal segmentation includes the detection and identification of one or more retinal (e.g., retina-associated) elements in a retinal image. A retinal element may be comprised of at least one of a retinal layer element or a retinal pathological element. Detection and identification of one or more retinal layer elements may be referred to as layer element (or retinal layer element) segmentation. Detection and identification of one or more retinal pathological elements may be referred to as pathological element (or retinal pathological element) segmentation.

A retinal layer element may be, for example, a retinal layer or a boundary associated with a retinal layer. Examples of retinal layers include, but are not limited to, an internal limiting membrane (ILM) layer, a retinal nerve fiber layer, a ganglion cell layer, an inner plexiform layer, an inner nuclear layer, an outer plexiform layer, an outer nuclear layer, an external limiting membrane (ELM) layer, a photoreceptor layer(s), a retinal pigment epithelial (RPE) layer, a layer of RPE detachment, a Bruch's membrane (BM) layer, a choriocapillaris layer, a choroidal stroma layer, an ellipsoid zone (EZ), and other types of retinal layer. In some cases, a retinal layer may be comprised of one or more layers. As one example, a retinal layer may be an outer plexiform layer-Henle fiber layer (OPL-HFL). A boundary associated with a retinal layer may be, for example, an inner boundary of the retinal layer, an outer boundary of the retinal layer, a boundary associated with a pathological feature of the retinal layer (e.g., an inner or outer boundary of detachment of the retinal layer), or some other type of boundary. For example, a boundary may be an inner boundary of an RPE (IB-RPE) detachment layer, an outer boundary of the RPE (OB-RPE) detachment layer, or another type of boundary.

A retinal pathological element may include, for example, fluid (e.g., a fluid pocket), cells, solid material, or a combination thereof that evidences a retinal pathology (e.g., disease or condition such as AMD or DME). For example, the presence of certain retinal fluids may be a sign of nAMD or DME. Examples of retinal pathological elements include, but are not limited to, intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, drusen, a development of fibrosis, and a disruption. In some cases, a retinal pathological element may be a disruption (e.g., discontinuity, delamination, loss, etc.) of a retinal layer or retinal zone. For example, the disruption may be of the ellipsoid zone, of the ELM, of the RPE, or of another layer or zone. The disruption may represent damage to or loss of cells (e.g., photoreceptors) in the area of the disruption.

Additionally, a retinal pathological element may include a characteristic or subtype of one of the fluids (e.g., IRF, SRF, fluid associated with PED), materials (e.g., HRM, SHRM, IHRM), lesions (e.g., HRF, SHRM lesions), or disruptions. In particular, examples of retinal pathological elements may include characteristics and/or subtypes of the different types of elements and disruptions described above that can be detected and identified via retinal segmentation. For example, whether a retinal fluid is clear or turbid may be detectable and identifiable characteristic of the retinal fluid. Accordingly, in some examples, a retinal pathological element may be clear IRF, turbid IRF, clear SRF, turbid SRF, some other type of clear retinal fluid, some other type of turbid retinal fluid, or a combination thereof. In some cases, for SHRM, shape characteristics (e.g., tall SHRM, dome-shaped SHRM at the foveal center, flat SHRM near the foveal center, dysmorphic, etc.), boundary characteristics (e.g., ill-defined SHRM, well-defined SHRM), reflectivity (e.g., increased reflectivity or other levels of reflectivity), layering characteristics (e.g., hyperreflective bands in SHRM lesions), and lesion characteristics (e.g., the height, width, and/or area of SHRM lesions) may be examples of retinal pathological elements that may be detected and identified via retinal segmentation.

Existing methodologies and systems for performing retinal segmentation may be more time-consuming than desired. For example, some currently available methodologies require manual annotation of images (e.g., a human grader detecting retinal elements and annotating the images to identify the retinal elements). This type of process may be tedious, may be prone to human error, and may be a bottleneck that increases the overall time and effort needed for overall image analysis in the detection, diagnosis, and/or treatment of an ophthalmological disease or condition. Further, in some cases, manual grading of pathologies via images may be too cumbersome or otherwise infeasible for human graders (e.g., when there are hundreds or thousands of images to grade, when there are very small objects scattered across an image, etc.). Further, manual grading by human graders may introduce undesired bias or variability to the grading.

Some currently available methodologies use computer processing to perform segmentation of retinal layers and to perform segmentation of retinal fluids, but these methodologies are less accurate than desired. For example, some currently available methodologies use algorithms built into OCT imaging devices that are less reliable than desired. These algorithms may be unable to, for example, accurately perform retinal segmentation in cases of atrophy with choroidal hypertransmission. Further, using the data generated by retinal segmentation algorithms included within OCT imaging devices provided by different vendors may cause issues because different vendors have different definitions for central subfield thickness (CST). Accordingly, the CST measurements generated using one OCT imaging device may not be comparable with the CST measurements generated using another OCT imaging device.

Thus, the embodiments described herein provide methodologies and systems for performing automated retinal segmentation of retinal elements in a manner that improves accuracy and reduces processing times. In particular, the methodologies and systems disclosed herein relate to automated retinal segmentation of retinal scans based on algorithms that use machine learning. The embodiments described herein enable the grading and retinal segmentation of much larger quantities of images and across the entirety of the images more accurately and efficiently than is possible with currently available methodologies and systems. Further, the embodiments described herein enable a finer level of detail in retinal segmentation because retinal segmentation is performed at the pixel level. The embodiments described herein also provide more predictability and reliability because overall bias and variability is reduced.

The disclosed methodologies and systems use machine learning to automatically perform retinal segmentation of OCT images. An OCT image may take the form of, but is not limited to, a time domain optical coherence tomography (TD-OCT) image, a spectral domain optical coherence tomography (SD-OCT) image, a two-dimensional OCT image, a three-dimensional OCT image, an OCT angiography (OCT-A) image, or a combination thereof. Although SD-OCT, also known as Fourier domain OCT, may be referred to with respect to the embodiments described herein, other types of OCT images are also contemplated for use with the methodologies and systems described herein. Thus, the description of embodiments with respect to images, image types, and techniques provides merely non-limiting examples of such images, image types, and techniques.

In one or more embodiments, one or more OCT images are processed to automatically perform retinal segmentation and generate one or more segmented OCT images. A segmented OCT image identifies one or more retinal elements on the segmented OCT image using one or more graphical indicators. For example, one or more color indicators, shape indicators, pattern indicators, shading indicators, lines, curves, markers, labels, tags, text features, other types of graphical indicators, or a combination thereof may be used to identify the portion(s) (e.g., by pixel) of an OCT image that have been identified as a retinal element.

As one specific example, a group of pixels may be identified as capturing a particular retinal fluid (e.g., IRF or SRF). A segmented OCT image may identify this group of pixels using a color indicator. For example, each pixel of the group of pixels may be assigned a color that is unique to the particular retinal fluid and thereby assigns each pixel to the particular retinal fluid. As another example, the segmented OCT image may identify the group of pixels by applying a patterned region or shape (continuous or discontinuous) over the group of pixels.

The segmented OCT image may be used to extract feature data for the one or more retinal elements identified in the segmented OCT image. The feature data may include values for any number of or combination of features (e.g., quantitative features). Examples of such features may include, but are not limited to, a maximum retinal layer thickness, a minimum retinal layer thickness, an average retinal layer thickness, a maximum height of a boundary associated with a retinal layer, a volume of a retinal fluid pocket, a length of a fluid pocket, a width of a fluid pocket, a number of retinal fluid pockets, a height of a lesion (e.g., SHRM lesion), a width of a lesion, an area of a lesion, a computed reflectivity (e.g., a reflectivity category or score for an SHRM lesion), and a number of hyperreflective foci.

The methodologies and systems described herein use retinal images, such as OCT images, to identify retinal elements to detect, diagnose, and/or treat an ophthalmological disease, such as AMD, diabetic retinopathy (DR), or DME. For example, first, an OCT image is generated (or captured) using a retina scanner or another type of OCT imaging device. The OCT image may be a TD-OCT image, an SD-OCT image, or some other type of OCT image. In other examples, the OCT image is received (or acquired) from the retina scanner (or other OCT imaging device) or from another source (e.g., data storage, a computer, etc.). Once obtained, the OCT image is processed using an algorithm that includes one or more artificial intelligence (AI)-based machine learning (ML) algorithms to perform retinal segmentation. For example, the algorithm may use neural networks to process the OCT image and perform layer element segmentation and pathological element segmentation.

The methodologies and systems described herein use layer element segmentation to generate layer element data that is used to refine the pathological element data generated by pathological element segmentation. For example, the OCT image may be processed via two pathways, each of which may be implemented using one or more neural networks. A first pathway includes performing automated layer element segmentation to generate layer element data such as, for example, a layer element image. The layer element image is a segmented OCT image that identifies a set of retinal layer elements using one or more graphical indicators (which may be referred to as layer element indicators). A second pathway includes performing automated pathological element segmentation to generate pathological element data such as, for example, a pathological element image. The pathological element image is a segmented OCT image that identifies a set of retinal pathological elements using one or more graphical indicators (which may be referred to as pathological element indicators). In one or more embodiments, the second pathway includes using the layer element data generated via the first pathway to refine the pathological element data generated along the second pathway such that the refined pathological element data more accurately identifies and locates the set of retinal pathological elements.

This type of refining of the pathological element image based on the layer element image ensures more accurate pathological element segmentation, which in turn, ensures more accurate detection, diagnosis, and/or treatment. For example, this type of refining may enable automatically correcting for imaging artifacts and/or defects to improve accuracy and reduce or prevent false-positive results that would otherwise occur, as with previous methods of processing. In addition to improving the accuracy with which a computer system is able to perform retinal segmentation, using the layer element image generated using neural network processing to refine the pathological element image generated using neural network processing may reduce overall processing times for retinal segmentation and thus, overall times for detection, diagnosis, and/or treatment.

Recognizing and taking into account the importance and utility of a methodology and system that can provide the improvements described above, the specification describes various embodiments for performing automated retinal segmentation, which may include layer element segmentation and pathological element segmentation, using a ML-based algorithm. The embodiments described herein enable more accurate and more reliable retinal segmentation, which may improve the accuracy and reliability of any detection, diagnosis, and/or treatment methodologies that rely on the results of this retinal segmentation.

### II. Machine Learning (ML)-Based Retinal Segmentation

### II.A. Example Systems for Automated Retinal Segmentation

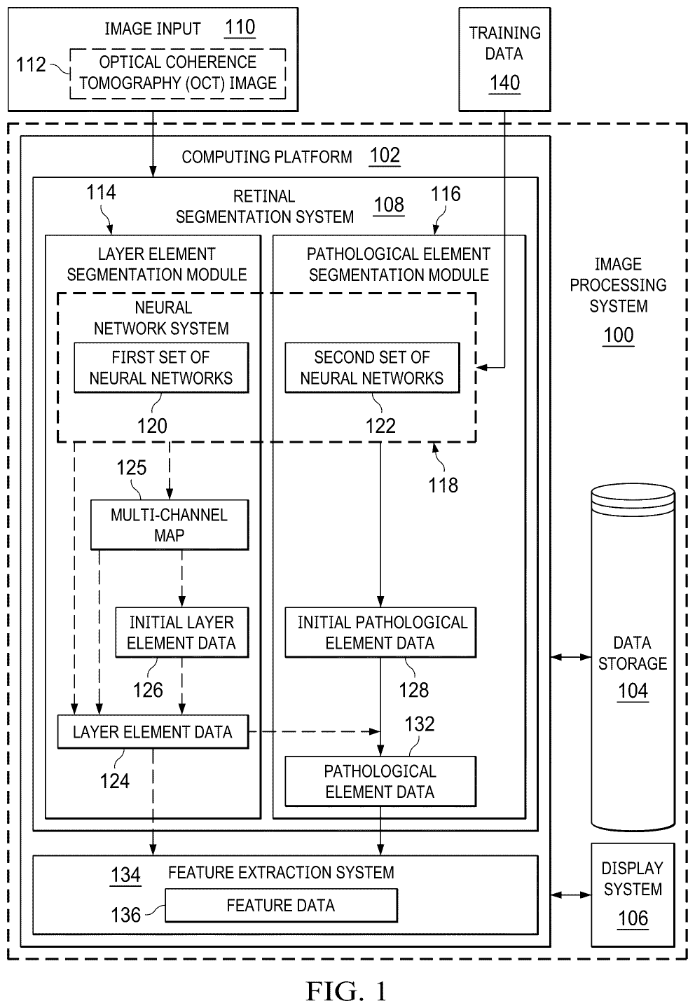

FIG. 1 is a block diagram of an image processing system 100, in accordance with various embodiments. The image processing system 100 is used for automatically performing retinal segmentation of retinal images to aid in the evaluation, detection, diagnosis, and/or treatment of patients with one or more ophthalmological diseases (or conditions) such as, for example, but not limited to, nAMD, DME, and DR. Image processing system 100 can include a computing platform 102, a data storage 104, and a display system 106. Computing platform 102 may take various forms. In one or more embodiments, computing platform 102 includes a single computer (or computer system) or multiple computers in communication with each other. In other examples, computing platform 102 takes the form of a cloud computing platform, a mobile computing platform (e.g., a smartphone, a tablet, etc.), or a combination thereof.

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

The image processing system 100 includes retinal segmentation system 108, which may be implemented using hardware, software, firmware, or a combination thereof. In one or more embodiments, retinal segmentation system 108 is implemented in computing platform 102. Retinal segmentation system 108 is used to perform automated retinal segmentation of input 110 that is received for processing. Input 110 may be received from another computing platform, retrieved from a database, uploaded from a cloud computing platform, received via an electronic message (e.g., email), received from a data storage device, retrieved from a data structure, or received in some other manner. In one or more embodiments, input 110 is retrieved from data storage 104.

Input 110 may include image input such as, for example, one or more retinal images. In one or more embodiments, input 110 includes OCT image(s) 112. OCT image 112 may be, for example, an SD-OCT image or a TD-OCT image of the retina of a subject who is experiencing and/or has been diagnosed with an ophthalmological disease (e.g., AMD, DR, or DME).

In some embodiments, input 110 may additionally include one or more color fundus (CF) images, one or more fundus autofluorescence (FAF) images, one or more fluorescein angiography (FA) images, one or more other types of OCT images (e.g., OCT-A images), one or more other types of retinal images, or a combination thereof. In this manner, input 110 may include multi-modal image input. Using multi-modal image input may increase the accuracy of the retinal segmentation.

Retinal segmentation system 108 includes layer element segmentation module 114 and pathological element segmentation module 116, each of which may be implemented using software, firmware, hardware, or a combination thereof. In one or more embodiments, layer element segmentation module 114 and pathological element segmentation module 116 are separate modules that work together to perform automated retinal segmentation. In other embodiments, layer element segmentation module 114 and pathological element segmentation module 116 may be integrated together within a single module. Layer element segmentation module 114 and pathological element segmentation module 116 are used in two different pathways of processing.

Layer element segmentation module 114 is used to perform layer element segmentation to detect and identify retinal layer elements. As previously described in Section I, a retinal layer element may be, for example, a retinal layer or a boundary associated with a retinal layer. Examples of retinal layers include, but are not limited to, an internal limiting membrane (ILM) layer, an external limiting membrane (ELM) layer, an outer plexiform layer-Henle fiber layer (OPL-HFL), a retinal pigment epithelial (RPE) layer, a layer of RPE detachment, a Bruch's membrane (BM) layer, an ellipsoid zone (EZ), and other types of retinal layers. A boundary associated with a retinal layer may be, for example, an inner boundary of the retinal layer, an outer boundary of the retinal layer, a boundary associated with a pathological feature of the retinal layer (e.g., an inner or outer boundary of detachment of the retinal layer), or some other type of boundary. For example, a boundary may be an inner boundary of an RPE (IB-RPE) detachment layer, an outer boundary of the RPE (OB-RPE) detachment layer, or another type of boundary.

Pathological element segmentation module 116 is used to perform pathological element segmentation to detect and identify retinal pathological elements. As previously described in Section I, a retinal pathological element may include, for example, fluid, cells, solid material, or a combination thereof that evidences a retinal pathology associated with an ophthalmological disease or condition. For example, the presence of certain retinal fluids may be a sign of leakage from retinal blood vessels, which may be a sign of nAMD. As another example, the presence of certain retinal fluids, like intraretinal fluid, may be a sign of DME. Examples of retinal pathological elements include, but are not limited to, intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, and a disruption. In some cases, a retinal pathological element may be a disruption (e.g., discontinuity, delamination, loss, etc.) of a retinal layer or retinal zone. For example, the disruption may be of the ellipsoid zone, of the ELM, of the RPE, or of another layer or zone. The disruption may represent damage to or loss of cells (e.g., photoreceptors) in the area of the disruption.

Additionally, a retinal pathological element may include a characteristic or subtype of one of the fluids (e.g., IRF, SRF, fluid associated with PED), materials (e.g., HRM, SHRM, IHRM), lesions (e.g., HRF, SHRM lesions), or disruptions. In particular, examples of retinal pathological elements may include characteristics and/or subtypes of the different types of elements and disruptions described above that can be detected and identified via retinal segmentation. For example, whether a retinal fluid is clear or turbid may be detectable and identifiable characteristic of the retinal fluid. Accordingly, in some examples, a retinal pathological element may be clear IRF, turbid IRF, clear SRF, turbid SRF, some other type of clear retinal fluid, some other type of turbid retinal fluid, or a combination thereof. In some cases, for SHRM, shape characteristics (e.g., tall SHRM, dome-shaped SHRM at the foveal center, flat SHRM near the foveal center, dysmorphic, etc.), boundary characteristics (e.g., ill-defined SHRM, well-defined SHRM), reflectivity (e.g., increased reflectivity or other levels of reflectivity), layering characteristics (e.g., hyperreflective bands in SHRM lesions), and lesion characteristics (e.g., the height, width, and/or area of SHRM lesions) may be examples of retinal pathological elements that may be detected and identified via retinal segmentation.

In some cases, a retinal layer element is associated with a retinal pathological element. For example, an RPE detachment layer, which is a retinal layer element, is associated with PED, which is a retinal pathological element. Accordingly, layer element segmentation module 114 and pathological element segmentation module 116 may communicate with each other in order to automatically and more accurately perform retinal segmentation.

In one or more embodiments, retinal segmentation system 108 uses a machine learning system to perform the automated segmentation. The machine learning system may include, for example, a deep learning system such as, but not limited to, neural network system 118. Neural network system 118 may include any number of or combination of neural networks. In one or more embodiments, neural network system 118 takes the form of a convolutional neural network (CNN) system that includes one or more convolutional neural networks. For example, the CNN may include a plurality of neural networks, each of which may itself be a convolutional neural network.

In one or more embodiments, a first portion of neural network system 118 is implemented within layer element segmentation module 114, while a second portion of neural network system 118 is implemented within pathological element segmentation module 116. For example, layer element segmentation module 114 may include a first neural network 120 of neural network system 118; pathological element segmentation module 116 may include a second neural network 122 of neural network system 118.

Each of first neural network 120 and second neural network 122 may be itself comprised of a set of neural networks. In one or more embodiments, first neural network 120 and second neural network 122 differ by at least one neural network. In other words, second neural network 122 may include at least one neural network that is different from the one or more neural networks in first neural network 120. In other embodiments, first neural network 120 and second neural network 122 may include the same one or more types of neural networks. For example, the same one or more types of neural networks may be used to perform both layer element segmentation and pathological element segmentation. In some cases, first neural network 120, second neural network 122, or both may include one or more mathematical algorithms or functions in addition to a set of neural networks.

In one or more embodiments, input 110 is processed along a first pathway using layer element segmentation module 114, which uses first neural network 120 to perform automated layer element segmentation. For example, layer element segmentation module 114 may receive input 110 (e.g., OCT image 112) at first neural network 120 for processing. In some embodiments, layer element segmentation module 114 preprocesses input 110 to enable focused attention on particular regions of interest prior to inputting input 110 into first neural network 120. This preprocessing may include, for example, reducing noise and/or artifacts in input 110 that might otherwise impair the ability to properly assess particular regions of interest. In some cases, first neural network 120 is trained to preprocess input 110.

Layer element segmentation module 114 uses first neural network 120 to process the input received (e.g., input 110 or the preprocessed image input) to perform automated layer element segmentation and generate layer element data 124 for a set of retinal layer elements detected within input 110 or the preprocessed image input. Layer element data 124 may include, for example, without limitation, a layer element image (which may also be referred to as a layer element segmented image), pixel data that assigns each pixel or section of pixels to a retinal layer element, image coordinates that map out each retinal layer element, other information for the set of retinal layer elements that have been detected, or a combination thereof.

A layer element image, which may be a layer element OCT image, includes a set of graphical indicators, which may be referred to as a set of layer element indicators. The set of layer element indicators identifies a set of retinal layer elements. A layer element indicator may take the form of, for example, without limitation, a color indicator, a shape indicator, a pattern indicator, a shading indicator, a line, a curve, a marker, a label, a tag, text, another type of graphical indicator, or a combination thereof. In some cases, two or more layer element indicators may identify a same retinal layer element. For example, a particular color may be used to identify pixels that represent a particular retinal layer element, while a label may be used to name or identify the particular retinal layer element associated with the particular color.

In some examples, a layer element indicator for identifying a retinal layer element that is a boundary associated with a retinal layer takes the form of a colored and/or patterned curve (continuous or discontinuous) on the layer element image. This curve represents the boundary. In other examples, a layer element indicator for identifying a retinal layer element that is a retinal layer may take the form of a colored and/or patterned region or shape (continuous or discontinuous) on the layer element image. The region or shape may represent, for example, the full thickness of the corresponding retinal layer.

In some embodiments, first neural network 120 receives input 110 (or preprocessed image input) and generates multi-channel map 125, which is then used to generate layer element data 124. Multi-channel map 125 may be comprised of a plurality of segmented images, with each segmented image of the plurality of segmented images corresponding to a different retinal layer element or a different retinal layer of interest. As one example, the plurality of segmented images may include a different segmented image for each retinal layer element of interest. As another example, the plurality of segmented images may include a different segmented image for each retinal layer of interest. In some cases, there may be two or more retinal layer elements of interest corresponding to a same retinal layer (e.g., an inner boundary and an outer boundary for the same retinal layer).

First neural network 120 may output multi-channel map 125 and layer segmentation module 114 may further process multi-channel map 125 using any number of or combination of various mathematical techniques (e.g., curve approximation, logistic function(s), smoothing function(s), another type of function or algorithm, or a combination thereof) to generate layer element data 124. In other embodiments, multi-channel map 125 may be produced as an intermediate output by first neural network 120, which then uses multi-channel map 125 to generate layer element data 124 as the output of first neural network 120.

In still other embodiments, multi-channel map 125 may be processed to generate initial layer element data 126 that is then refined to form layer element data 124 (which can then be referred to as refined layer element data). Initial layer element data 126 may include, but is not limited to, a layer element image (which may also be referred to as a layer element segmented image), pixel data that assigns each pixel or section of pixels to a retinal layer element, image coordinates that map out each retinal layer element, other information of the set of retinal layer elements that have been detected, or a combination thereof. But in these examples, initial layer element data 126 may be a first approximation.

As one example, initial layer element data 126 may include an initial layer element image having at least one layer element indicator that identifies a boundary associated with a retinal layer of interest. This initial layer element image may be processed using any number of or combination of various mathematical techniques (e.g., curve approximation, smoothing function(s), another type of function or algorithm, or a combination thereof) to refine the initial layer element image and generate a refined layer element image that forms at least a portion of layer element data 124. In one or more embodiments, this refinement may be a smoothing of the identified boundary.

In other embodiments, initial layer element data 126 may be produced as an intermediate output by first neural network 120, which then uses initial layer element data 126 to generate layer element data 124 as the output of first neural network 120. In this manner, layer element data 124 may be generated in any number of different ways by layer element segmentation module 114 within the first pathway of processing.

In one or more embodiments, input 110 is also processed along a second pathway using pathological element segmentation module 116, which uses second neural network 122 of neural network system 118 to perform pathological element segmentation. For example, pathological element segmentation module 116 may receive input 110 (e.g., OCT image 112) at second neural network 122 for processing. In some embodiments, pathological element segmentation module 116 preprocesses input 110 to enable focused attention on particular regions of interest prior to inputting input 110 into second neural network 122. This preprocessing may include, for example, reducing noise and/or artifacts in input 110 that might otherwise impair the ability to properly assess particular regions of interest. In some cases, second neural network 122 is trained to preprocess input 110.

Pathological element segmentation module 116 uses second neural network 122 to process the input received (e.g., input 110 or the preprocessed image input) to perform automated pathological element segmentation and generate initial pathological element data 128 for a set of pathological layer elements detected within input 110 or the preprocessed image input. Initial pathological element data 128 may include, for example, without limitation, a pathological element image (which may also be referred to as a pathological element segmented image), pixel data that assigns each pixel or section of pixels to a retinal pathological element, image coordinates that map out each retinal pathological element, other information for the set of retinal pathological elements that have been detected, or a combination thereof.

A pathological element image, which may be a pathological element OCT image, includes a set of graphical indicators, which may be referred to as a set of pathological element indicators. The set of pathological element indicators identifies a set of retinal pathological elements. A pathological element indicator may take the form of, for example, without limitation, a color indicator, a shape indicator, a pattern indicator, a shading indicator, a line, a curve, a marker, a label, a tag, text, another type of graphical indicator, or a combination thereof. In some cases, two or more pathological element indicators may identify a same retinal pathological element. For example, a particular color may be used to identify pixels that represent a particular retinal pathological element, while a label may be used to name or identify the particular retinal pathological element associated with the particular color.

In some examples, a pathological element indicator for identifying a retinal pathological element that is a retinal fluid may take the form of a colored and/or patterned region or shape (continuous or discontinuous) on the pathological element image. The region or shape may represent, for example, the pocket formed by the retinal fluid.

Initial pathological element data 128 output from second neural network 122 may then be further processed and refined by pathological element segmentation module 116. For example, pathological element segmentation module 116 receives layer element data 124 (or at least a portion of layer element data 124) from layer element segmentation module 114. Pathological element segmentation module 116 uses both initial pathological element data 128 and layer element data 124 to refine initial pathological element pathological 128 and generate pathological element data 132, which may be referred to as refined pathological element data.

Similar to initial pathological element data 128, pathological element data 132 may include, for example, without limitation, a pathological element image (which may also be referred to as a pathological element segmented image), pixel data that assigns each pixel or section of pixels to a retinal pathological element, image coordinates that map out each retinal pathological element, other information for the set of retinal pathological elements that have been detected, or a combination thereof. Pathological element data 132 more accurately identifies and locates the set of retinal pathological elements that are of interest as compared to initial pathological element data 128. For example, the pathological element data 132 includes a refined pathological element image with a set of pathological element indicators, this set of pathological element indicators may more accurately identify at least one corresponding retinal pathological element as compared to initial pathological element data 128.

In one or more embodiments, pathological element segmentation module 116 uses layer element data 124 to constrain the allowable area for the set of retinal pathological elements identified in pathological element data 132. For example, a portion of layer element data 124 corresponding to two retinal layers may be used to constrain the allowable area for a retinal pathological element such that the retinal pathological element is not identified as extending beyond the allowable area for the retinal pathological element. As one specific example, layer element data 124 may be used to constrain the allowable area for an intraretinal fluid in the pathological element image such that the intraretinal fluid is not identified by a corresponding pathological element indicator as crossing over into a subretinal space.

Thus, layer element data 124 can be used to refine the anatomic characterization of a retinal pathological element of the set of retinal pathological elements identified in pathological element data 132 using the one or more pathological element indicators that correspond to the retinal pathological element. The anatomic characterization of a retinal pathological element may include at least one of, for example, without limitation, the location, size, shape, length, width, thickness, volume, or other characteristic of the retinal pathological element.

In other embodiments, initial pathological element data 128 may be an intermediate output of second neural network 122 and layer element data 124 may be input into second neural network 122 to refine initial pathological element data 128. In these examples, second neural network 122 outputs pathological element data 132.

Refining initial pathological element data 128 using layer element data 124 improves the overall accuracy of pathological element segmentation module 116 generating pathological element data 132. This improvement in accuracy may be carried through in any future analysis conducted using pathological element data 132.

For example, feature extraction system 134 may be implemented in computing platform 102. Feature extraction system 134 may be used to automatically extract feature data 136 from pathological element data 132 and, in some cases, layer element data 124. Feature data 136 may include values for any number of or combination of features (e.g., quantitative features). Examples of such features may include, but are not limited to, a maximum retinal layer thickness, a minimum retinal layer thickness, an average retinal layer thickness, a maximum height of a boundary associated with a retinal layer, a volume of a retinal fluid pocket, a length of a fluid pocket, a width of a fluid pocket, a number of retinal fluid pockets, and a number of hyperreflective foci.

Refining initial pathological element data 128 to form (refined) pathological element data 132 improves the accuracy of feature data 136 that is extracted. Further, any detection, diagnosis, and/or treatment methodologies that rely on pathological element data 132 and/or feature data 136 extracted from pathological element data 132 may be more accurate.

In one or more embodiments, feature data 136 includes values for features that are associated with the ETDRS (Early Treatment of Diabetic Retinopathy Score) grid. The ETDRS grid divides the retina into nine regions defined by two rings and a central region. The central region represents the foveal center. The two rings include the inner macular ring and the outer macular ring. The inner macular ring is divided into four regions: a superior inner region, a temporal inner region, an inferior inner region, and a nasal inner region. The outer macular ring is divided into four regions: a superior outer region, a temporal outer region, an inferior outer region, and a nasal outer region.

In one or more embodiments, a value for a feature (e.g., a number of fluid pockets, a volume of fluid, etc.) may be generated with respect to the foveal center, the inner macular ring, or the outer macular ring. A value for a feature may be generated with respect to a particular region (e.g., quadrant) of the inner macular ring or outer macular ring. In some cases, a value for a feature may be generated with respect to two corresponding regions of the two rings (e.g., the superior inner region of the inner macular ring and the superior outer region of the outer macular ring). Thus, a value for a feature may be generated for any single region of the ETDRS grid, for a ring of the ETDRS grid, for a multi-region area formed by multiple regions of the ETDRS grid, or the central region of the ETDRS grid. More accurate retinal segmentation, as provided by the embodiments described herein, allows more accurate extraction of feature data with respect to the various regions and multi-region areas of the ETDRS grid.

In one or more embodiments, neural network system 118 is trained using training data 140. For example, first neural network 120 may be trained using a first training dataset of training data 140, while second neural network 122 may be trained using a second training dataset of training data 140. The first training dataset may include, for example, without limitation, a plurality of training OCT images and training layer element data (e.g., a plurality of training layer element images). The second training dataset may include a plurality of training OCT images (which may be the same as, partially the same as, or different from the plurality of training OCT images in the first training dataset) and training pathological element data (e.g., a plurality of training pathological element images).

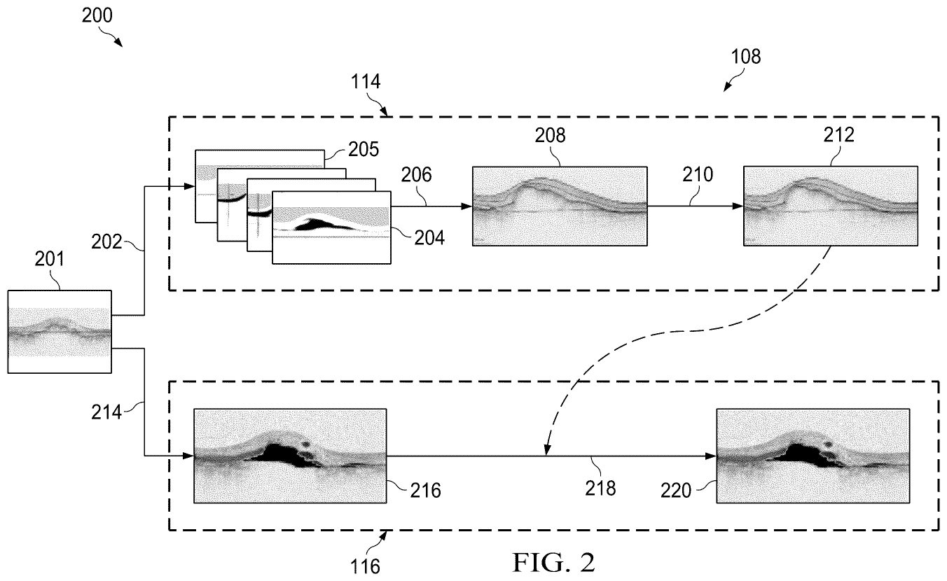

FIG. 2 is a schematic diagram of an example workflow 200 for performing automated retinal segmentation using an OCT image, in accordance with various embodiments. Workflow 200 is one example of an implementation for automated retinal segmentation that may be performed using retinal segmentation system 108 in FIG. 1. For example, workflow 200 may be implemented using layer element segmentation module 114 and pathological element segmentation module 116 in FIG. 1.

Retinal segmentation system 108 receives input 201 for processing. In one or more embodiments, input 201 includes an OCT image (e.g., OCT image 112 in FIG. 1). In some embodiments, the OCT image may be a preprocessed OCT image. Input 201 may be sent into a first pathway of processing that uses layer element segmentation module 114 and a second pathway of processing that uses pathological element segmentation module 116.

Layer element segmentation module 114 receives input 201 and processes input 201 via neural network operation 202. Neural network operation 202 may be implemented using first neural network 120. In one or more embodiments, first neural network 120 includes a CNN such as, for example, but not limited to, a U-Net for performing forward prediction.

Layer element segmentation module 114 may use at least a portion of first neural network 120 to process input 201 via neural network operation 202 and generate multi-channel map 204. Multi-channel map 204 is one example of an implementation for multi-channel map 125 in FIG. 1. Multi-channel map 204 includes a plurality of segmented images 205.

In one or more embodiments, each segmented image of the plurality of segmented images 205 corresponds to a different retinal layer. For example, a different segmented image may be generated for each different retinal layer that is of interest. Further, each segmented image may identify the corresponding retinal layer of interest using at least one graphical indicator (e.g., a color indicator, a shape indicator, a pattern indicator, a shading indicator, a marker, a label, a tag, text, another type of graphical indicator, or a combination thereof). The one or more graphical indicators, which may be referred to as layer element indicators, visually identify the portion of the segmented image that represents the corresponding retinal layer of interest. For example, a group of pixels that represent the corresponding retinal layer of interest may be assigned to the corresponding retinal layer of interest and visually identified via a color indicator. The coloring of this group of pixels may visually identify the region (continuous or discontinuous) of the segmented image that represents the corresponding retinal layer of interest.

Layer element segmentation module 114 processes multi-channel map 204 via a curve approximation operation 206 to generate an initial layer element image 208. Initial layer element image 208 may be one example of an implementation for initial layer element data 126 in FIG. 1. Curve approximation operation 206 may include performing, for example, a piecewise logistic curve approximation to approximate at least one boundary associated with each retinal layer of interest identified in multi-channel map 204. In one or more embodiments, a boundary associated with a retinal layer may be the inner boundary (e.g., the anatomically innermost boundary) for the retinal layer.

When the region that represents the corresponding retinal layer of interest in a segmented image of multi-channel map 204 is discontinuous (e.g., formed by multiple regions separated by gaps), curve approximation operation 206 approximates a continuous or near-continuous boundary (e.g., inner boundary, outer boundary, etc.) that extends across the discontinuous region. In this manner, curve approximation operation 206 may be used to identify a single continuous or near continuous boundary for the corresponding retinal layer of interest that is identified in initial layer element image 208 using at least one graphical indicator (e.g., a colored and/or patterned line that highlights the boundary in initial layer element image 208).

In one or more embodiments, one or more boundaries are identified for each retinal layer of interest identified in multi-channel map 204 and identified on initial layer element image 208 using any number of layer element indicators. In this manner, multi-channel map 204 comprised of a plurality of segmented images 205 may be processed to form a single initial layer element image 208. When initial layer element image 208 identifies such boundaries (e.g., as opposed to full thicknesses of retinal layers), initial layer element image 208 may be referred to as an elevation map.

Additionally, layer element segmentation module 114 may process initial layer element image 208 via smoothing operation 210 to generate refined layer element image 212. Refined layer element image 212 is one example of an implementation for layer element data 124 in FIG. 1. Refined layer element image 212 includes a set of layer element indicators that more accurately identify the locations of the boundaries within refined layer element image 212 as compared to initial layer element image 208. Smoothing operation 210 may be performed using, for example, n-dimensional Gaussian smoothing. This smoothing helps smooth the curves generated via curve approximation operation 206 in initial layer element image 208, reduce noise, or both to generate refined layer element image 212.

In a second pathway of processing, pathological element segmentation module 116 receives input 201 and processes input 201 via neural network operation 214. Neural network operation 214 may be implemented using second neural network 122. In one or more embodiments, second neural network 122 includes a CNN such as, for example, but not limited to, a U-Net for performing forward prediction.

Pathological element segmentation module 116 may use at least a portion of second neural network 122 to process input 201 via neural network operation 214 and generate initial pathological element image 216. Initial pathological element image 216 is one example of an implementation for initial pathological element data 128 in FIG. 1.

Initial pathological element image 216 identifies a set of retinal pathological elements using one or more graphical indicators (e.g., a color indicator, a shape indicator, a pattern indicator, a shading indicator, a marker, a label, a tag, text, another type of graphical indicator, or a combination thereof). The one or more graphical indicators, which may be referred to as pathological element indicators, visually identify the one or more portions of initial pathological element image 216 that have been identified as representing the set of retinal pathological elements of interest. For example, a group of pixels that represents a retinal pathological element of interest may be assigned to that retinal pathological element and visually identified via a color indicator. The coloring of this group of pixels may visually identify the region (continuous or discontinuous) of initial pathological element image 216 that represents the retinal pathological element. This identification is an approximation.

Pathological element segmentation module 116 proceeds to refine initial pathological element image 216 using refined layer element image 212. For example, pathological element segmentation module 116 may receive refined layer element image 212 from layer element segmentation module 114. Pathological element segmentation module 116 uses refined layer element image 212 to perform refining operation 218 on initial pathological element image 216 and thereby generate refined pathological element image 220. Refined pathological element image 220 may be one example of an implementation for pathological element data 132 in FIG. 1. Refined pathological element image 220 identifies the set of retinal pathological elements using one or more pathological element indicators more accurately than initial pathological element image 216.

Refining operation 218 may refine initial pathological element image 216 by, for example, constraining the allowable area for the set of retinal pathological elements using refined layer element image 212 (or data extracted from refined layer element image 212). For example, one or more boundaries identified in refined layer element image 212 may be used to constrain the allowable area for a retinal pathological element such that the retinal pathological element is not identified as extending beyond the allowable area for the retinal pathological element. As one specific example, one or more boundaries in refined layer element image 212 may be used to constrain the allowable area for an intraretinal fluid such that the intraretinal fluid is not identified by a corresponding pathological element indicator as crossing over into a subretinal space in refined pathological element image 220. In this manner, refining operation 218 ensures that the anatomic characterization of the set of retinal pathological elements in refined pathological element image 220 using the set of pathological element indicators is accurate (e.g., anatomically feasible, clinically relevant, and/or otherwise proper).

Accordingly, the retinal segmentation system 108 described in FIGS. 1 and 2 illustrates a system for automated and reliable identification of retinal layer elements and retinal pathological elements (e.g., nAMD-related retinal elements, DME-related retinal elements, etc.) in OCT images of a retina. Improved accuracy in retinal segmentation may enable more accurate and/or clinically relevant diagnostic and/or treatment solutions for patients afflicted with, for example, nAMD, DR, DME, or other ophthalmological diseases or conditions.

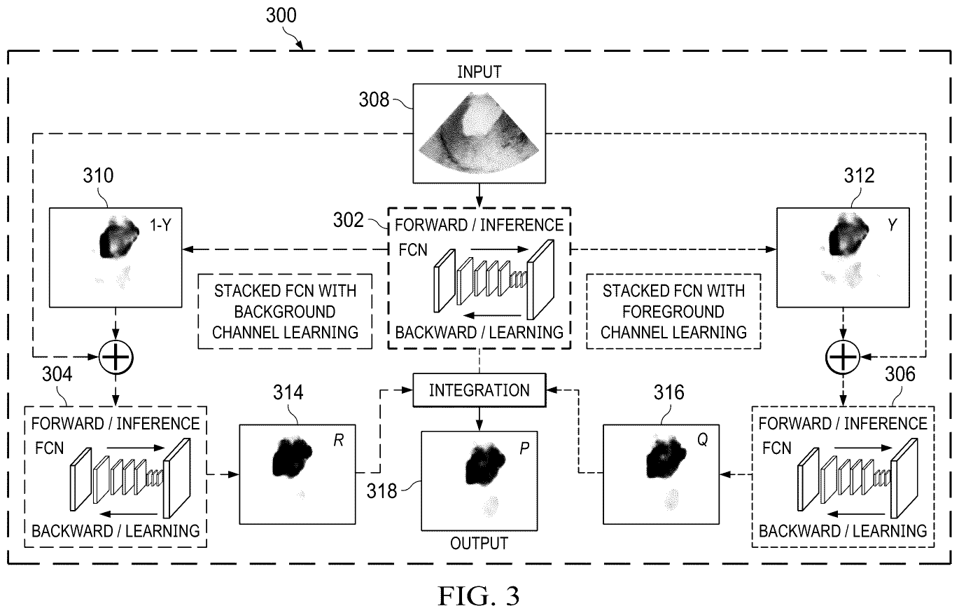

FIG. 3 is a schematic diagram illustrating a neural network that can be used in retinal segmentation system 108 in FIG. 1, in accordance with various embodiments. Neural network 300 is one example of an implementation for a neural network in neural network system 118 in FIG. 1 that can be implemented within retinal segmentation system 108 in FIG. 1. In particular, neural network 300 may be one example of an implementation for a neural network in first neural network 120 in FIG. 1 or one example of an implementation for second neural network 122 in FIG. 1. Neural network 300 may be used in performing automated layer element segmentation. For example, neural network system 300 may be used to generate a multi-channel map, such as multi-channel map 125 in FIG. 1 or multi-channel map 204 in FIG. 2.

Neural network 300 may include initial neural network 302, background neural network 304, and foreground neural network 306. In one or more embodiments, each of initial neural network 302, background neural network 304, and foreground neural network 306 may be implemented as a fully convolutional network (FCN) (e.g., a stacked FCN). In other embodiments, neural network 300 may include one or more other types or combinations of neural networks.

Initial neural network 302 receives image input 308 for processing. Image input 308 may be one example of an implementation for image input included in input 110 in FIG. 1 or input 201 in FIG. 2. Image input 308 takes the form of an OCT image (e.g., OCT image 112 in FIG. 1). In some cases, the OCT image may be the image received directly from a retinal scanner or other type of OCT imaging device or may be a preprocessed OCT image. Initial neural network 302 processes image input 308 to generate background probability map 310 and foreground probability map 312.

Background probability map 310 identifies (or segments out) a background of image input 308. In one or more embodiments, this background may be anything in image input 308 that is not of interest. For example, the background may be any portion of image input 308 that is not a retinal layer of interest. In one or more embodiments, background probability map 310 includes a separate background probability image for each retinal layer of interest such that a background probability image for a corresponding retinal layer of interest identifies the background of the image with respect to the corresponding retinal layer of interest using at least one graphical indicator.

As one example, background probability map 310 may include a first background probability image and a second background probability image. The first background probability image identifies a background with respect to a first retinal layer of interest by coloring (or shading, patterning, etc.) the group of pixels that is identified as representing the background differently from the rest of the pixels in the image. The second background probability image identifies background with respect to a second retinal layer of interest by coloring (or shading, patterning, etc.) the group of pixels that is identified as representing the background differently from the rest of the pixels in the image.

Foreground probability map 312 identifies (or segments out) a foreground of image input 308. In one or more embodiments, this foreground may be anything in image input 308 that is of interest. For example, the foreground may be any portion of image input 308 that represents a retinal layer of interest. In one or more embodiments, foreground probability map 312 includes a separate foreground probability image for each retinal layer of interest such that a foreground probability image for a corresponding retinal layer of interest identifies the corresponding retinal layer of interest using at least one graphical indicator.

As one example, foreground probability map 312 may include a first foreground probability image and a second foreground probability image. The first foreground probability image identifies a first retinal layer of interest by coloring (or shading, patterning, etc.) the group of pixels that is identified as representing the first retinal layer of interest differently from the rest of the pixels in the image. The second foreground probability image identifies a second retinal layer of interest by coloring (or shading, patterning, etc.) the group of pixels that is identified as representing the second retinal layer of interest differently from the rest of the pixels in the image.

Background probability map 310 and image input 308 are combined and sent as input into background neural network 304 to generate refined background map 314. Refined background map 314 more accurately identifies (segments out) the portion(s) of image input 308 that do not represent a retinal layer of interest. For example, refined background map 314 may include a plurality of refined background images, each of which identifies a background of the image with respect to a respective retinal layer of interest more accurately than the corresponding background probability image in background probability map 310.

Foreground probability map 312 and image input 308 are combined and sent as input into foreground neural network 306 to generate refined foreground map 316. Refined foreground map 316 more accurately identifies (segments out) the portion(s) of image input 308 that represents one or more retinal layers of interest. For example, refined foreground map 316 may include a plurality of refined foreground images, each of which identifies a corresponding retinal layer of interest more accurately than the corresponding foreground probability image in foreground probability map 312.

Refined background map 314 and refined foreground map 316 are then integrated to form a multi-channel map 318. This integration may be performed using a concatenating and/or stacking operation of both maps. Multi-channel map 318 may be one example of an implementation for multi-channel map 125 in FIG. 1 or multi-channel map 204 in FIG. 2. In one or more embodiments, multi-channel map 318 includes a separate segmented image for each retinal layer of interest. In other words, each segmented image clearly and accurately identifies the portion of that image that represents a corresponding retinal layer of interest.

### II.B. Example Methodologies for Automated Retinal Segmentation

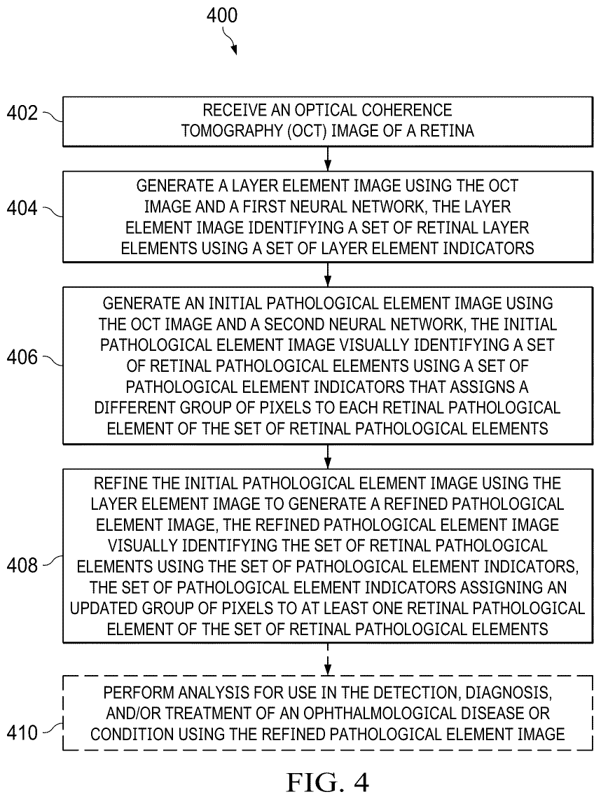

FIG. 4 is a flowchart of a method 400 of performing retinal segmentation, in accordance with various embodiments. In various embodiments, the method 400 can be implemented using the image processing system 100 described in FIG. 1. For example, method 400 may be implemented using retinal segmentation system 108 described with respect to FIGS. 1 and 2. In one or more embodiments, a portion of method 400 may be implemented using neural network 300 in FIG. 3.

As illustrated in FIG. 4, the method 400 includes, at step 402, receiving an optical coherence tomography (OCT) image of a retina of a subject. The subject may be afflicted with an ophthalmological disease or condition. For example, the subject may be experiencing and/or diagnosed with AMD (e.g., nAMD), DR, DME, or another ophthalmological disease or condition. In various embodiments, the OCT image may be OCT image 112 of the input 110 as described with respect to FIG. 1. The OCT image may be, for example, an SD-OCT image or a TD-OCT image.

The method 400 further includes, at step 404, generating a layer element image using the OCT image and a first neural network, the layer element image identifying a set of retinal layer elements using a set of layer element indicators. A retinal layer element may be, for example, a retinal layer or a boundary associated with a retinal layer. A retinal layer may be, for example, but is not limited to, an internal limiting membrane (ILM) layer, an external limiting membrane (ELM) layer, an ellipsoid zone (EZ), an outer plexiform layer-Henle fiber layer (OPL-HFL), a retinal pigment epithelial (RPE) layer, a layer of RPE detachment, a Bruch's membrane (BM) layer, or another type of retinal layer. A boundary associated with a retinal layer may be, for example, an inner boundary of the retinal layer, an outer boundary of the retinal layer, a boundary associated with a pathological feature of the retinal layer (e.g., an inner or outer boundary of detachment of the retinal layer), or some other type of boundary. For example, a boundary may be an inner boundary of an RPE (IB-RPE) detachment layer, an outer boundary of the RPE (OB-RPE) detachment layer, or another type of boundary.

The set of layer element indicators used in layer element image may be a set of graphical indicators. For example, a layer element indicator may be, for example, but is not limited to, a color indicator, a shape indicator, a pattern indicator, a shading indicator, a line, a curve, a marker, a label, a tag, text, or another type of graphical indicator.

In one or more embodiments, the layer element image visually identifies one or more portions of the layer element image that have been identified as representing a retinal layer element of interest. The retinal layer element of interest may be, for example, a boundary associated with a retinal layer. In one or more embodiments, the layer element image may visually identify this retinal layer of interest by assigning a group of pixels that represent the boundary to a color that has been assigned to that retina boundary. When each retinal layer element of interest in the layer element image is a boundary, the layer element image may be referred to as an elevation map.

Step 404 may be performed using a first neural network, such as first neural network 120 in FIG. 1. The first neural network may include, for example, without limitation, at least one of a CNN, an FCN, a stacked FCN, a stacked FCN with multi-channel learning, a U-Net, or another type of neural network. In one or more embodiments, the first neural network is used to perform all of the operations involved in step 404. In other embodiments, the first neural network is used to perform a portion of the operations involved in step 404.

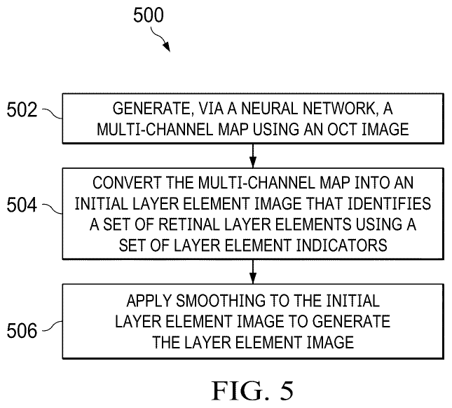

Step 404 may be performed in various ways. Method 500 in FIG. 5 below is one example of a method that may be used to implement step 404.

The method 400 further includes, at step 406, generating an initial pathological element image using the OCT image and a second neural network, the initial pathological element image visually identifying a set of retinal pathological elements using a set of pathological element indicators that assigns a different group of pixels to each retinal pathological element of the set of retinal pathological elements. This identification may be an approximation.

A retinal pathological element may include, for example, fluid, cells, solid material, or a combination thereof that evidences a retinal pathology associated with an ophthalmological disease or condition. For example, the presence of certain retinal fluids may be a sign of leakage from retinal blood vessels, which may be a sign of nAMD. As another example, the presence of certain retinal fluids, such as intraretinal fluid, may be a sign of DME. Examples of retinal pathological elements include, but are not limited to, intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, and a disruption. In some cases, a retinal pathological element may be a disruption (e.g., discontinuity, delamination, loss, etc.) of a retinal layer or retinal zone. For example, the disruption may be of the ellipsoid zone, of the ELM, of the RPE, or of another layer or zone. The disruption may represent damage to or loss of cells (e.g., photoreceptors) in the area of the disruption.

Additionally, a retinal pathological element may include a characteristic or subtype of one of the fluids (e.g., IRF, SRF, fluid associated with PED), materials (e.g., HRM, SHRM, IHRM), lesions (e.g., HRF, SHRM lesions), or disruptions. In particular, examples of retinal pathological elements may include characteristics and/or subtypes of the different types of elements and disruptions described above that can be detected and identified via retinal segmentation. For example, whether a retinal fluid is clear or turbid may be detectable and identifiable characteristic of the retinal fluid. Accordingly, in some examples, a retinal pathological element may be clear IRF, turbid IRF, clear SRF, turbid SRF, some other type of clear retinal fluid, some other type of turbid retinal fluid, or a combination thereof. In some cases, for SHRM, shape characteristics (e.g., tall SHRM, dome-shaped SHRM at the foveal center, flat SHRM near the foveal center, dysmorphic, etc.), boundary characteristics (e.g., ill-defined SHRM, well-defined SHRM), reflectivity (e.g., increased reflectivity or other levels of reflectivity), layering characteristics (e.g., hyperreflective bands in SHRM lesions), and lesion characteristics (e.g., the height, width, and/or area of SHRM lesions) may be examples of retinal pathological elements that may be detected and identified via retinal segmentation.

In one or more embodiments, the initial pathological element image visually identifies one or more portions of the initial pathological element image that have been identified as representing a retinal pathological element of interest. The retinal pathological element of interest may be, for example, subretinal fluid. In one or more embodiments, the initial pathological element image visually identifies the subretinal fluid by assigning a group of pixels that represent the subretinal fluid to a color that has been assigned to the subretinal fluid.

Step 406 may be performed using a second neural network, such as second neural network 122 in FIG. 1. The second neural network may include, for example, without limitation, at least one of a CNN, an FCN, a stacked FCN, a stacked FCN with multi-channel learning, a U-Net, or another type of neural network. In one or more embodiments, the second neural network is used to perform all of the operations involved in step 406. In other embodiments, the second neural network is used to perform a portion of the operations involved in step 406.

The method 400 further includes, at step 408 refining the initial pathological element image using the layer element image to generate a refined pathological element image, the refined pathological element image visually identifying the set of retinal pathological elements using the set of pathological element indicators, the set of pathological element indicators assigning an updated group of pixels to at least one retinal pathological element of the set of retinal pathological elements. The refined pathological element image more accurately represents at least one retinal pathological element of the set of retinal pathological elements as compared to the initial pathological element image.

The refining in step 408 may be performed in different ways. For example, the refining in step 408 includes updating a group of pixels in the initial pathological element image that is assigned to a particular retinal pathological element to form the updated group of pixels for the retinal pathological element in the refined pathological element image by constraining an allowable area for the retinal pathological element based on the layer element image. The allowable area may be constrained based on what is anatomically feasible, clinically relevant, and/or otherwise proper. The updated group of pixels includes fewer pixels than the group of pixels.

In one or more embodiments, a pathological element indicator of the set of pathological element indicators may be used to assign a group of pixels in the initial pathological element image to a first retinal pathological element of the set of retinal pathological elements. Refining the initial pathological element image may include reassigning a portion of the group of pixels in the initial pathological element image based on whether an anatomical characterization of the first retinal pathological element as identified by the pathological element indicator is anatomically feasible. The anatomical characterization of the first retinal pathological element of the set of retinal pathological elements may include at least one of a location, a size, a shape, a length, a width, a thickness, a volume of the retinal pathological element, or another characteristic.

In one or embodiments, the reassigning of the portion of the group of pixels may include, for example, reassigning a first pixel of the group of pixels from the first retinal pathological element to a second retinal pathological element of the set of retinal pathological elements based on the layer element image. Reassigning a pixel to a different retinal pathological element may include, for example, without limitation, changing the application of a pathological element indicator associated with that pixel. For example, the pixel may be changed from a first color in the initial pathological element image to a second color in the refined pathological element image.

In one or embodiments, the reassigning of the portion of the group of pixels may include, for example, reassigning a second pixel of the group of pixels from the first retinal pathological element to a background based on the layer element image. Reassigning a pixel to background may include, for example, without limitation, removing the application of a pathological element indicator associated with that pixel. For example, a color that was previously applied to that pixel in the initial pathological element image may be removed in the refined pathological element image.

The above examples of reassigning pixels are merely illustrative and are not meant to pose any limitations to the manner in which pixels may be reassigned. The reassigning of pixels in step 408 may be performed based on whether the anatomical characterization of the set of retinal pathological elements as presented by the set of pathological element indicators in initial pathological element image is allowable (e.g., anatomically feasible, clinically relevant, and/or otherwise proper). For example, a pixel annotated with a particular pathological element indicator that assigns that pixel to a particular retinal pathological element may be reassigned if the location of that pixel makes it anatomically infeasible to be associated with the particular retinal pathological element. Such determinations are made using the layer element image and/or data extracted from the layer element image.

The method 400 may optionally include, at step 410, performing analysis for use in the detection, diagnosis and/or treatment of an ophthalmological disease or condition using the refined pathological element image. The ophthalmological disease or condition may be, for example, nAMD, DME, or DR. The analysis in step 410 may include, for example, extracting feature data from the refined pathological element image and in some cases, from the layer element image. The feature data may include values for any number of or combination of features (e.g., quantitative features). Examples of such features may include, but are not limited to, a maximum retinal layer thickness, a minimum retinal layer thickness, an average retinal layer thickness, a maximum height of a boundary associated with a retinal layer, a volume of a retinal fluid pocket, a length of a fluid pocket, a width of a fluid pocket, a number of retinal fluid pockets, and a number of hyperreflective foci.

In various embodiments, the first neural network described in step 404, the second neural network described in step 406, or both may be trained using training data such as training data 140 in FIG. 1. The first neural network may be trained using, for example, a first training dataset comprising a first plurality of training OCT images and a plurality of training layer element images. The plurality of training layer element images may include training multi-channel maps, training initial layer element images, training refined layer element images, or a combination thereof.

FIG. 5 is a flowchart of a method 500 for generating a layer element image, in accordance with various embodiments. In various embodiments, the method 500 can be implemented using the image processing system 100 described in FIG. 1. For example, the method 500 may be implemented using retinal segmentation system 108 described with respect to FIGS. 1 and 2. In one or more embodiments, the method 500 may be implemented using neural network 300 in FIG. 3. The method 500 may be one example of a method that can be used to implement step 404 in FIG. 4. The method 500 may include one or more steps or operations of workflow 200 in FIG. 2.

As illustrated in FIG. 5, the method 500 includes, at step 502, generating, via a neural network, a multi-channel map using an OCT image. The multi-channel map comprises a plurality of segmented images in which each segmented image of the plurality of segmented images identifies a corresponding retinal layer of interest. The multi-channel map may be, for example, multi-channel map 125 in FIG. 1, multi-channel map 204 in FIG. 2, or multi-channel map 318 in FIG. 3. The OCT image, which may be the OCT image received in step 402 in process 400 in FIG. 4, may be, for example, OCT image 112 in FIG. 1. The neural network may be, for example, first neural network 120 in FIG. 1 or neural network 300 in FIG. 3. In one or more embodiments, the neural network may include at least one of a CNN, an FCN, a stacked FCN, a stacked FCN with multi-channel learning, a U-Net, or another type of neural network.

The method 500 further includes, at step 504, converting the multi-channel map into an initial layer element image that identifies a set of retinal layer elements using a set of layer element indicators. The set of layer element indicators assigns a different group of pixels in the initial layer element image to each retinal layer element of the set of retinal layer elements. The conversion in step 504 may be performed by, for example, applying piecewise logistic curve approximation to the multi-channel map to generate the initial layer elevation. The set of retinal layer elements may related to the various retinal layers identified in multi-channel map 204. In some cases, two or more retinal layer elements may correspond with a same retinal layer of interest.

The method 500 further includes, at step 506, applying smoothing to the initial layer element image to generate the layer element image. This smoothing may be performed using, for example, Gaussian smoothing (e.g., n-dimensional (n-D) Gaussian smoothing). In one or more embodiments, the initial layer element image and the layer element image both take the form of elevation maps.

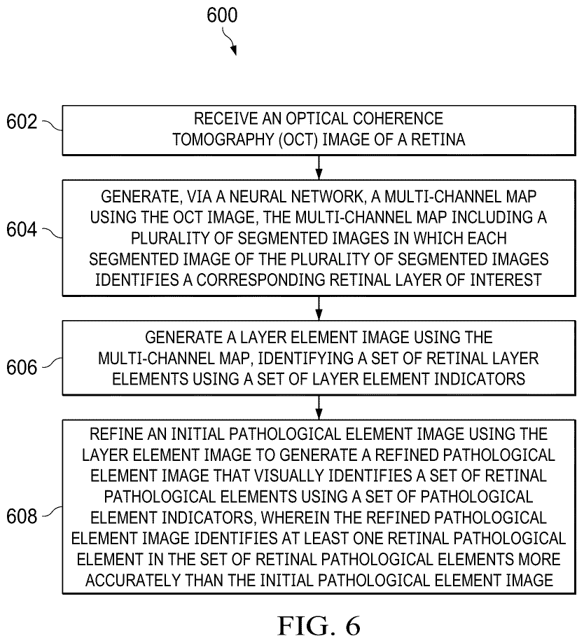

FIG. 6 is a flowchart of another method 600 for performing retinal segmentation, in accordance with various embodiments. In various embodiments, the method 600 can be implemented using the image processing system 100 described in FIG. 1. For example, method 600 may be implemented using retinal segmentation system 108 described with respect to FIGS. 1 and 2. In one or more embodiments, a portion of method 600 may be implemented using neural network 300 in FIG. 3.

The method 600 includes, at step 602, receiving an optical coherence tomography (OCT) image of a retina. The OCT image may be, for example, OCT image 112 in FIG. 1.

The method 600 further includes, at step 604, generating, via a neural network, a multi-channel map using the OCT image, the multi-channel map including a plurality of segmented images in which each segmented image of the plurality of segmented images identifies a corresponding retinal layer of interest. In one or more embodiments, the neural network includes one or more fully convolutional networks (FCNs). In one or more embodiments, the neural network includes a U-Net. The neural network may be, for example, neural network 300 in FIG. 3.

The method 600 further includes, at step 606, generating a layer element image using the multi-channel map, identifying a set of retinal layer elements using a set of layer element indicators. In one or more embodiments, step 606 includes converting the multi-channel map into an initial layer element image that identifies boundaries associated with the retinal layers of interest identified by the multi-channel map. In some cases, a boundary associated with a retinal layer of interest estimates an inner boundary of the retinal layer. The conversion in step 606 may be performed by applying piecewise logistic curve approximation to the multi-channel map to generate the initial layer element image. In some embodiments, step 606 includes applying smoothing to the initial layer elevation map to generate the layer element image. In other embodiments, the initial layer element image is used as the layer element image.

The method 600 may further include, at step 608, refining an initial pathological element image using the layer element image to generate a refined pathological element image that visually identifies a set of retinal pathological elements using a set of pathological element indicators. The refined pathological element image identifies at least one retinal pathological element in the set of retinal pathological elements more accurately than the initial pathological element image. The initial pathological element image may have been generated using a different neural network.

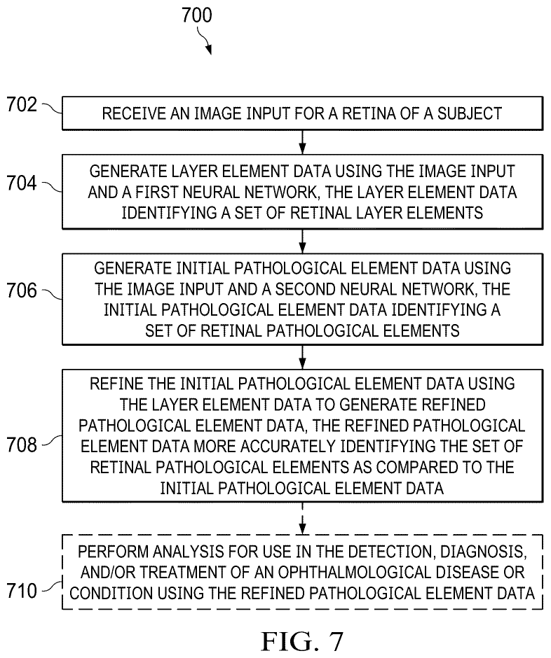

FIG. 7 is a flowchart of another method 700 for performing automated retinal segmentation, in accordance with various embodiments. In various embodiments, the method 700 can be implemented using the image processing system 100 described in FIG. 1. For example, method 700 may be implemented using retinal segmentation system 108 described with respect to FIGS. 1 and 2.

Step 702 includes receiving an image input for a retina of a subject. The image input may be, for example, input 201 in FIG. 2 (or input 110 in FIG. 1). The image input may include an OCT image (e.g., an SD-OCT image).

Step 704 includes generating layer element data using the image input and a first neural network, the layer element data identifying a set of retinal layer elements. The layer element data may be, for example, layer element data 124 in FIG. 1. In one or more embodiments, the layer element data comprises a layer element image that identifies a set of retinal layer elements using a set of layer element indicators.

A retinal layer element of the set of retinal layer elements is either a retinal layer or a boundary associated with the retinal layer. The retinal layer may be, for example, but is not limited to, an internal limiting membrane (ILM) layer, an external limiting membrane (ELM) layer, an outer plexiform layer-Henle fiber layer (OPL-HFL), a retinal pigment epithelial (RPE) layer, a layer of RPE detachment, a Bruch's membrane (BM) layer, an ellipsoid zone (EZ), or another type of retinal layer.

Step 706 includes generating initial pathological element data using the image input and a second neural network, the initial pathological element data identifying a set of retinal pathological elements. The initial pathological element data may be, for example, initial pathological element data 128 in FIG. 1. In one or more embodiments, the initial pathological element data comprises an initial pathological element image that visually identifies the set of retinal pathological elements using a set of pathological element indicators that assigns a different group of pixels in the initial pathological element image to each retinal pathological element of the set of retinal pathological elements.

The set of retinal pathological elements includes at least one of intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, or a disruption. In some cases, a retinal pathological element may be a disruption (e.g., discontinuity, delamination, loss, etc.) of a retinal layer or retinal zone. For example, the disruption may be of the ellipsoid zone, of the ELM, of the RPE, or of another layer or zone. The disruption may represent damage to or loss of cells (e.g., photoreceptors) in the area of the disruption.

Step 708 includes refining the initial pathological element data using the layer element data to generate refined pathological element data, the refined pathological element data more accurately identifying the set of retinal pathological elements as compared to the initial pathological element data. The refined pathological element data may be, for example, refined pathological element data 132 in FIG. 1. In one or more embodiments, the refined pathological element data comprises a refined pathological element image that visually identifies the set of retinal pathological elements using the set of pathological element indicators, the set of pathological element indicators assigning an updated group of pixels to at least one retinal pathological element of the set of retinal pathological elements.

Step 710 may optionally include performing an analysis for use in the detection, diagnosis, and/or treatment of an ophthalmological disease or condition (e.g., nAMD, DR, or DME) using the refined pathological element data. The analysis in step 710 may include, for example, extracting feature data from the refined pathological element data and in some cases, from the layer element data. The feature data may include values for any number of or combination of features (e.g., quantitative features). Examples of such features may include, but are not limited to, a maximum retinal layer thickness, a minimum retinal layer thickness, an average retinal layer thickness, a maximum height of a boundary associated with a retinal layer, a volume of a retinal fluid pocket, a length of a fluid pocket, a width of a fluid pocket, a number of retinal fluid pockets, and a number of hyperreflective foci.

In one or more embodiments, a retinal pathological element may be a biomarker for one or more ophthalmological diseases or conditions. For example, the detection of the retinal pathological element may indicate the presence of the one or more ophthalmological diseases or conditions. The refinement in step 708 improves the accuracy of any disease detection and/or diagnosis conducted based on the identification of a retinal pathological element via the refined pathological element data. In some embodiments, performing the refinement in step 708 helps improve the accuracy of the analysis conducted in step 710 and thereby, improves the accuracy of any detection, diagnosis, and/or treatment methods or solutions based on this analysis.

### III. Example Results of the Systems and Methods Disclosed Herein

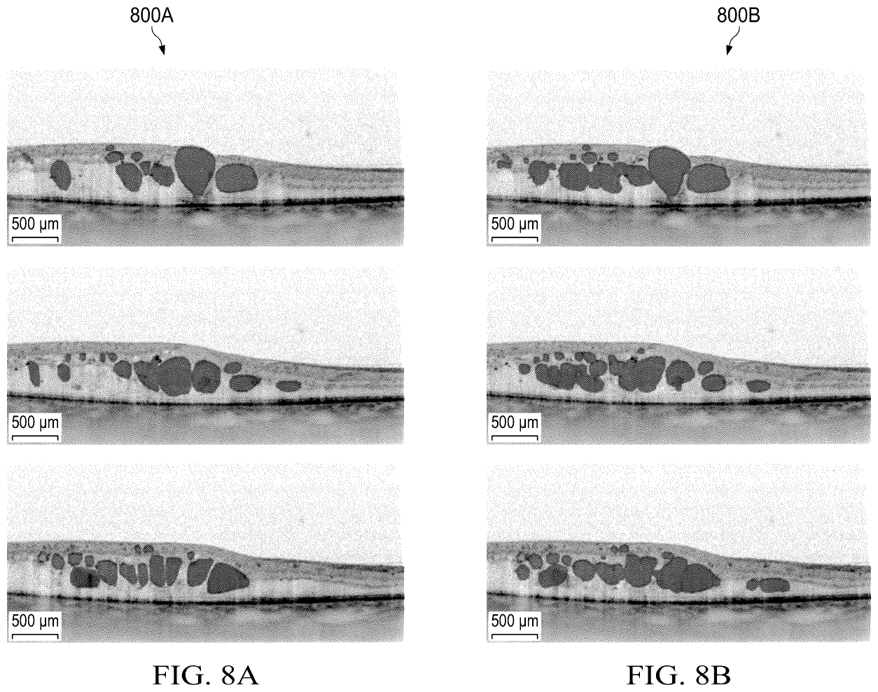

FIGS. 8A and 8B are illustrations of retinal segmentation results in accordance with various embodiments. FIG. 8A is an illustration of manual retinal segmentation results 800A in accordance with various embodiments. FIG. 8B is an illustration of automated (e.g., automated ML-based) retinal segmentation results 800B in accordance with various embodiments.

Manual retinal segmentation results 800A are based on annotations performed by an expert, such as, Liverpool Reading Center, per their standard operating procedures. In contrast, automated retinal segmentation results 800B are generated via an automated retinal segmentation system, such as retinal segmentation system 108 described with respect to FIGS. 1 and 2. Comparing manual retinal segmentation results 800A with automated retinal segmentation results 800B validates that the embodiments disclosed herein are capable of providing accurate and reliable results using ML-based algorithms. Additionally, the embodiments disclosed herein may be used to automatically correct image artifacts and/or defects. In some cases, the embodiments described herein provide a complete automated diagnostic solution for nAMD based on automated detection of retinal pathological elements that are known to be associated with nAMD. In other cases, the embodiments described herein may provide a complete automated diagnostic solution for other ophthalmological diseases or conditions (e.g., DR, DME) based on automated detection of retinal pathological elements that are known to be associated with such ophthalmological diseases or conditions.

### IV. Artificial Neural Networks

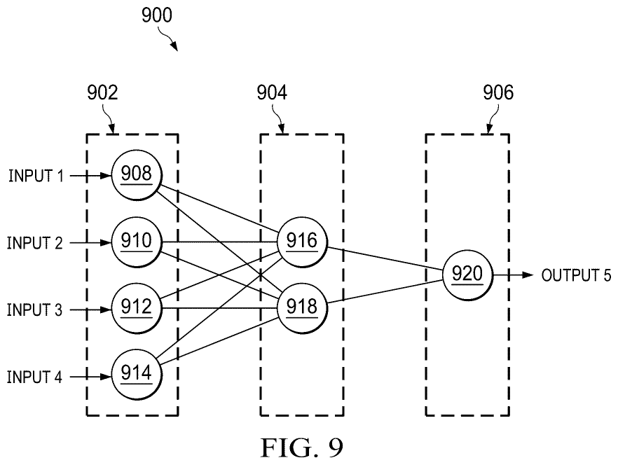

FIG. 9 is a schematic diagram of an example neural network that can be used to implement a computer-based model in accordance with various embodiments. For example, neural network 900 may be one example of an implementation for a neural network that may be included in first neural network 120, second neural network 122, or both in FIG. 1.

As shown, neural network 900 includes three layers—an input layer 902, a hidden layer 904, and an output layer 906. Each of input layer 902, hidden layer 904, and output layer 906 may include one or more nodes. In this example, input layer 902 includes node 908, node 910, node 912, and node 914; hidden layer 904 includes node 916 and node 918; and output layer 906 includes node 920.

In this example, each node in a layer is connected to every node in an adjacent layer. For example, node 908 in input layer 902 is connected to both node 916 and node 918 in hidden layer 904. Similarly, node 916 in hidden layer 904 is connected to all of nodes 908, node 910, node 912, and node 914 in input layer 902, as well as to node 920 in output layer 906. Although only one hidden layer is shown for neural network 900, neural network 900 may include any number of hidden layers between input layer 902 and output layer 906.

In this example, neural network 900 receives a set of input values (e.g., inputs 1-4) and produces an output value (e.g., output 5). Each node in input layer 902 may correspond to a distinct input value. As one example, the set of input values may include a set of attributes for an image, such as OCT image 112 in FIG. 1. In this example, each node in input layer 902 may correspond to and receive a distinct attribute of the image.

In some embodiments, each of the node 916 and node 918 in hidden layer 904 generates a representation, which may include a mathematical computation (or algorithm) that produces a value based on the input values received from the node 908, node 910, node 912, and node 914. The mathematical computation may include assigning different weights to each of the data values received from the node 908, node 910, node 912, and node 914. The nodes 916 and 918 may include different algorithms and/or different weights assigned to the data variables from the node 908, node 910, node 912, and node 914 such that each of nodes 916 and 918 may produce a different value based on the same input values received from node 908, node 910, node 912, and node 914. In some embodiments, the weights that are initially assigned to the features (or input values) for each of nodes 916 and 918 may be randomly generated (e.g., using a computer randomizer). The values generated by the nodes 916 and 918 may be used by node 920 in output layer 906 to produce an output value for neural network 900.

Neural network 900 may be trained using training data. For example, the training data may include various OCT images. By providing training data to neural network 900, node 916 and node 918 in hidden layer 904 may be trained (adjusted) such that an optimal output is produced in output layer 906 based on the training data. By continuously providing different sets of training data and penalizing neural network 900 when the output of neural network 900 is incorrect, neural network 900 (and specifically, the representations of the nodes in hidden layer 904) may be trained (adjusted) to improve its performance in data classification. Adjusting neural network 900 may include adjusting the weights associated with each node in hidden layer 904.

Although the above discussions pertain to an artificial neural network as an example of machine learning, it is understood that other types of machine learning methods may also be suitable to implement the various aspects of the present disclosure. For example, support vector machines (SVMs) may be used to implement machine learning. SVMs are a set of related supervised learning methods used for classification and regression. An SVM training algorithm-which may be a non-probabilistic binary linear classifier—may build a model that predicts whether a new example falls into one category or another. As another example, Bayesian networks may be used to implement machine learning. A Bayesian network is an acyclic probabilistic graphical model that represents a set of random variables and their conditional independence with a directed acyclic graph (DAG). The Bayesian network could present the probabilistic relationship between one variable and another variable. Another example is a machine learning engine that employs a decision tree learning model to conduct the machine learning process. In some instances, decision trec learning models may include classification tree models, as well as regression trec models.

In some embodiments, the machine learning engine employs a Gradient Boosting Machine (GBM) model (e.g., XGBoost) as a regression tree model. Other machine learning techniques may be used to implement the machine learning engine, for example via Random Forest or Deep Neural Networks. Other types of machine learning algorithms are not discussed in detail herein for reasons of simplicity and it is understood that the present disclosure is not limited to a particular type of machine learning.

### V. Computer Implemented System

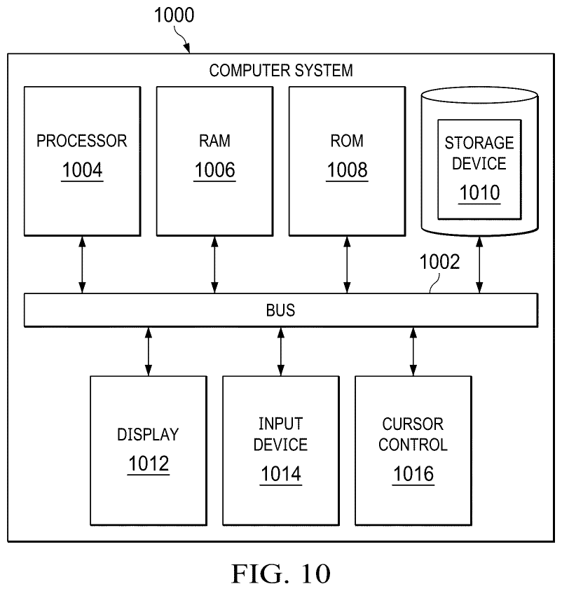

FIG. 10 is a block diagram of a computer system in accordance with various embodiments. Computer system 1000 may be an example of one implementation for computing platform 102 described above in FIG. 1. In one or more examples, computer system 1000 can include a bus 1002 or other communication mechanism for communicating information, and a processor 1004 coupled with bus 1002 for processing information. In various embodiments, computer system 1000 can also include a memory, which can be a random-access memory (RAM) 1006 or other dynamic storage device, coupled to bus 1002 for determining instructions to be executed by processor 1004. Memory also can be used for storing temporary variables or other intermediate information during execution of instructions to be executed by processor 1004. In various embodiments, computer system 1000 can further include a read only memory (ROM) 1008 or other static storage device coupled to bus 1002 for storing static information and instructions for processor 1004. A storage device 1010, such as a magnetic disk or optical disk, can be provided and coupled to bus 1002 for storing information and instructions.

In various embodiments, computer system 1000 can be coupled via bus 1002 to a display 1012, such as a cathode ray tube (CRT) or liquid crystal display (LCD), for displaying information to a computer user. An input device 1014, including alphanumeric and other keys, can be coupled to bus 1002 for communicating information and command selections to processor 1004. Another type of user input device is a cursor control 1016, such as a mouse, a joystick, a trackball, a gesture input device, a gaze-based input device, or cursor direction keys for communicating direction information and command selections to processor 1004 and for controlling cursor movement on display 1012. This input device 1014 typically has two degrees of freedom in two axes, a first axis (e.g., x) and a second axis (e.g., y), that allows the device to specify positions in a plane. However, it should be understood that input devices 1014 allowing for three-dimensional (e.g., x, y and z) cursor movement are also contemplated herein.

Consistent with certain implementations of the present teachings, results can be provided by computer system 1000 in response to processor 1004 executing one or more sequences of one or more instructions contained in RAM 1006. Such instructions can be read into RAM 1006 from another computer-readable medium or computer-readable storage medium, such as storage device 1010. Execution of the sequences of instructions contained in RAM 1006 can cause processor 1004 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 1004 for execution. Such a medium can take many forms, including but not limited to, non-volatile media, volatile media, and transmission media. Examples of non-volatile media can include, but are not limited to, optical, solid state, magnetic disks, such as storage device 1010. Examples of volatile media can include, but are not limited to, dynamic memory, such as RAM 1006. Examples of transmission media can include, but are not limited to, coaxial cables, copper wire, and fiber optics, including the wires that comprise bus 1002.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1004 of computer system 1000 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 1000 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1000, whereby processor 1004 would execute the analyses and determinations provided by these engines, subject to instructions provided by any one of, or a combination of, the memory components RAM 1006, ROM 1008, or storage device 1010 and user input provided via input device 1014.

### VI. Example Definitions and Context

The disclosure is not limited to these example embodiments and applications or to the manner in which the example embodiments and applications operate or are described herein. Moreover, the figures may show simplified or partial views, and the dimensions of elements in the figures may be exaggerated or otherwise not in proportion.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for case of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. In various cases, “subject” and “patient” may be used interchangeably herein.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “about” used with respect to numerical values or parameters or characteristics that can be expressed as numerical values means within ten percent of the numerical values. For example, “about 50” means a value in the range from 45 to 55, inclusive.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be used. The item may be a particular object, thing, step, operation, process, or category. In other words, “at least one of” means any combination of items or number of items may be used from the list, but not all of the items in the list may be used. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” may include the practice of using algorithms to parse data, learn from it, and then make a determination or prediction about something in the world. Machine learning may use algorithms that can learn from data without relying on rules-based programming. Deep learning may be one form of machine learning.

As used herein, an “artificial neural network” or “neural network” (NN) may refer to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks may include one or more hidden layers in addition to an output layer. The output of each hidden layer may be used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways; when it is being trained it is in training mode and when it puts what it has learned into practice it is in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may learn by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), a U-Net, a fully convolutional network (FCN), a stacked FCN, a stacked FCN with multi-channel learning, a Squeeze and Excitation embedded neural network, a MobileNet, or another type of neural network.

As used herein, “deep learning” may refer to the use of multi-layered artificial neural networks to automatically learn representations from input data such as images, video, text, etc., without human provided knowledge, to deliver highly accurate predictions in tasks such as object detection/identification, speech recognition, language translation, etc.

### VII. Additional Considerations

While the present teachings are described in conjunction with various embodiments, it is not intended that the present teachings be limited to such embodiments. On the contrary, the present teachings encompass various alternatives, modifications, and equivalents, as will be appreciated by those of skill in the art.

For example, the flowcharts and block diagrams described above illustrate the architecture, functionality, and/or operation of possible implementations of various method and system embodiments. Each block in the flowcharts or block diagrams may represent a module, a segment, a function, a portion of an operation or step, or a combination thereof. In some alternative implementations of an embodiment, the function or functions noted in the blocks may occur out of the order noted in the figures. For example, in some cases, two blocks shown in succession may be executed substantially concurrently. In other cases, the blocks may be performed in the reverse order. Further, in some cases, one or more blocks may be added to replace or supplement one or more other blocks in a flowchart or block diagram.

Thus, in describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### VIII. Recitation of Embodiments

Embodiment 1. A method for performing retinal segmentation, the method comprising: receiving an optical coherence tomography (OCT) image of a retina; generating a layer element image using the OCT image and a first neural network, the layer element image identifying a set of retinal layer elements using a set of layer element indicators; generating an initial pathological element image using the OCT image and a second neural network, the initial pathological element image visually identifying a set of retinal pathological elements using a set of pathological element indicators that assigns a different group of pixels to each retinal pathological element of the set of retinal pathological elements; and refining the initial pathological element image using the layer element image to generate a refined pathological element image, the refined pathological element image visually identifying the set of retinal pathological elements using the set of pathological element indicators, the set of pathological element indicators assigning an updated group of pixels to at least one retinal pathological element of the set of retinal pathological elements.

Embodiment 2. The method of embodiment 1, wherein a pathological element indicator of the set of pathological element indicators is used to assign a group of pixels in the initial pathological element image to a first retinal pathological element of the set of retinal pathological elements and wherein the refining comprises: reassigning a portion of the group of pixels in the initial pathological element image based on whether an anatomical characterization of the first retinal pathological element as identified by the pathological element indicator is anatomically feasible, wherein the anatomical characterization of the first retinal pathological element includes at least one of a location, a size, a shape, a length, a width, a thickness, or a volume of the retinal pathological element.

Embodiment 3. The method of embodiment 2, wherein the reassigning comprises at least one of: reassigning a first pixel of the group of pixels from the first retinal pathological element to a second retinal pathological element of the set of retinal pathological elements based on the layer element image; or reassigning a second pixel of the group of pixels from the first retinal pathological element to a background based on the layer element image.

Embodiment 4. The method of any one of embodiments 1-2, wherein the refining comprises: updating a group of pixels in the initial pathological element image assigned to a retinal pathological element of the set of retinal pathological elements to form the updated group of pixels for the retinal pathological element in the refined pathological element image by constraining an allowable area for the retinal pathological element based on the layer element image, wherein the updated group of pixels includes fewer pixels than the group of pixels.

Embodiment 5. The method of any one of embodiments 1-4, wherein generating the layer element image comprises: generating, via the first neural network, a multi-channel map using the OCT image, wherein the multi-channel map comprises a plurality of segmented images in which each segmented image of the plurality of segmented images identifies a corresponding retinal layer of interest.

Embodiment 6. The method of embodiment 5, wherein generating the layer element image further comprises: converting the multi-channel map into an initial layer element image that identifies the set of retinal layer elements using the set of layer element indicators, wherein the set of layer element indicators assigns a different group of pixels in the initial layer element image to each retinal layer element of the set of retinal layer elements.

Embodiment 7. The method of embodiment 6, wherein the converting comprises: applying piecewise logistic curve approximation to the multi-channel map to generate the initial layer element image.

Embodiment 8. The method of embodiment 6 or embodiment 7, wherein generating the layer element image further comprises: applying smoothing to the initial layer element image to generate the layer element image.

Embodiment 9. The method of embodiment 8, wherein applying smoothing to the initial layer element image comprises applying Gaussian smoothing to the initial layer element image to generate the layer element image.

Embodiment 10. The method of any one of embodiments 1-9, wherein a retinal layer element of the set of retinal layer elements is either a retinal layer or a boundary associated with the retinal layer.

Embodiment 11. The method of embodiment 10, wherein the retinal layer is selected from a group consisting of an internal limiting membrane (ILM) layer, an external limiting membrane (ELM) layer, an outer plexiform layer-Henle fiber layer (OPL-HFL), a retinal pigment epithelial (RPE) layer, a layer of RPE detachment, a Bruch's membrane (BM) layer, and an ellipsoid zone (EZ).

Embodiment 12. The method of any one of embodiments 10-11, wherein the set of retinal pathological elements includes at least one of intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, a disruption, or a characteristic or subtype of a fluid, material, or disruption.

Embodiment 13. The method of any one of embodiments 10-12, wherein each of the set of layer element indicators and the set of pathological element indicators includes at least one of a color indicator, a shape indicator, a pattern indicator, a shading indicator, a line, a curve, a marker, a label, a tag, or text.

Embodiment 14. The method of any one of embodiments 10-13, wherein the first neural network comprises a first U-Net and the second neural network comprises a second U-Net.

Embodiment 15. The method of any one of embodiments 10-14, wherein: the first neural network is trained using a first training dataset comprising a first plurality of training OCT images and a plurality of training layer element images; and the second neural network is trained using a second training dataset comprising a second plurality of training OCT images and a plurality of training pathological element images.

Embodiment 16. The method of embodiment 15, wherein at least a portion of the first plurality of training OCT images is included in the second plurality of training OCT images.

Embodiment 17. A method for performing retinal segmentation, the method comprising: receiving an optical coherence tomography (OCT) image of a retina; generating, via a neural network, a multi-channel map using the OCT image, the multi-channel map including a plurality of segmented images in which each segmented image of the plurality of segmented images identifies a corresponding retinal layer of interest; generating a layer element image using the multi-channel map, identifying a set of retinal layer elements using a set of layer element indicators; and refining an initial pathological element image using the layer element image to generate a refined pathological element image that visually identifies a set of retinal pathological elements using a set of pathological element indicators, wherein the refined pathological element image identifies at least one retinal pathological element in the set of retinal pathological elements more accurately than the initial pathological element image.

Embodiment 18. The method of embodiment 17, wherein generating the layer element image comprises: converting the multi-channel map into an initial layer element image using piecewise logistic curve approximation.

Embodiment 19. The method of embodiment 18, wherein generating the layer element image further comprises: applying smoothing to the initial layer element image to generate the layer element image that is then used to refine the initial pathological element image.

Embodiment 20. The method of any one of embodiments 17-19, wherein refining the initial pathological element image comprises at least one of: reassigning, based on the layer element image, a first portion of pixels in the initial pathological element image from one retinal pathological element to a different retinal pathological element in the refined pathological element image; or reassigning, based on the layer element image, a second portion of pixels in the initial pathological element image to a background in the refined pathological element image.

Embodiment 21. A system for performing automated retinal segmentation, comprising: a non-transitory memory; and a data processor coupled with the non-transitory memory and configured to read instructions from the non-transitory memory to cause the system to perform operations comprising: receiving an optical coherence tomography (OCT) image of a retina; generating a layer element image using the OCT image and a first neural network, the layer element image identifying a set of retinal layer elements using a set of layer element indicators; generating an initial pathological element image using the OCT image and a second neural network, the initial pathological element image visually identifying a set of retinal pathological elements using a set of pathological element indicators that assigns a different group of pixels to each retinal pathological element of the set of retinal pathological elements; and refining the initial pathological element image using the layer element image to generate a refined pathological element image, the refined pathological element image visually identifying the set of retinal pathological elements using the set of pathological element indicators, the set of pathological element indicators assigning an updated group of pixels to at least one retinal pathological element of the set of retinal pathological elements.

Embodiment 22. A method for performing automated retinal segmentation, the method comprising: receiving an image input for a retina of a subject; generating layer element data using the image input and a first neural network, the layer element data identifying a set of retinal layer elements; generating initial pathological element data using the image input and a second neural network, the initial pathological element data identifying a set of retinal pathological elements; and refining the initial pathological element data using the layer element data to generate refined pathological element data, the refined pathological element data more accurately identifying the set of retinal pathological elements as compared to the initial pathological element data.

Embodiment 23. The method of embodiment 22, wherein the initial pathological element data comprises an initial pathological element image that visually identifies the set of retinal pathological elements using a set of pathological element indicators that assigns a different group of pixels in the initial pathological element image to each retinal pathological element of the set of retinal pathological elements.

Embodiment 24. The method of embodiment 23, wherein the refined pathological element data comprises a refined pathological element image that visually identifies the set of retinal pathological elements using the set of pathological element indicators, the set of pathological element indicators assigning an updated group of pixels to at least one retinal pathological element of the set of retinal pathological elements.

Embodiment 25. The method of any one of embodiments 22-24, wherein the layer element data comprises a layer element image that identifies a set of retinal layer elements using a set of layer element indicators.

Embodiment 26. The method of any one of embodiments 22-25, wherein the image input comprises an SD-OCT image.

Embodiment 27. The method of any one of embodiments 22-26, wherein a retinal layer element of the set of retinal layer elements is either a retinal layer or a boundary associated with the retinal layer and wherein the retinal layer is selected from a group consisting of an internal limiting membrane (ILM) layer, an external limiting membrane (ELM) layer, an outer plexiform layer-Henle fiber layer (OPL-HFL), a retinal pigment epithelial (RPE) layer, a layer of RPE detachment, a Bruch's membrane (BM) layer, and an ellipsoid zone (EZ).

Embodiment 28. The method of any one of embodiments 22-27, wherein the set of retinal pathological elements includes at least one of intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, a disruption, or a characteristic or subtype of a fluid, material, or disruption.

Embodiment 29. A system comprising: one or more data processors; and a non-transitory computer readable storage medium containing instructions which, when executed on the one or more data processors, cause the one or more data processors to perform part or all of one or more methods disclosed in embodiments 1-20 and 22-28.

Embodiment 30. A computer-program product tangibly embodied in a non-transitory machine-readable storage medium, including instructions configured to cause one or more data processors to perform part or all of one or more methods disclosed in embodiments 1-20 and 22-28.

What is claimed is:

1. A method for performing retinal segmentation, the method comprising:
receiving an optical coherence tomography (OCT) image of a retina;
generating a layer element image using the OCT image and a first neural network, the layer element image identifying a set of retinal layer elements using a set of layer element indicators;
generating an initial pathological element image using the OCT image and a second neural network, the initial pathological element image visually identifying a set of retinal pathological elements using a set of pathological element indicators that assigns a different group of pixels to each retinal pathological element of the set of retinal pathological elements; and
refining the initial pathological element image using the layer element image to generate a refined pathological element image, the refined pathological element image visually identifying the set of retinal pathological elements using the set of pathological element indicators, the set of pathological element indicators assigning an updated group of pixels to at least one retinal pathological element of the set of retinal pathological elements.

2. The method of claim 1, wherein a pathological element indicator of the set of pathological element indicators is used to assign a group of pixels in the initial pathological element image to a first retinal pathological element of the set of retinal pathological elements and wherein the refining comprises:
reassigning a portion of the group of pixels in the initial pathological element image based on whether an anatomical characterization of the first retinal pathological element as identified by the pathological element indicator is anatomically feasible,
wherein the anatomical characterization of the first retinal pathological element includes at least one of a location, a size, a shape, a length, a width, a thickness, or a volume of the retinal pathological element.

3. The method of claim 2, wherein the reassigning comprises at least one of:
reassigning a first pixel of the group of pixels from the first retinal pathological element to a second retinal pathological element of the set of retinal pathological elements based on the layer element image; or
reassigning a second pixel of the group of pixels from the first retinal pathological element to a background based on the layer element image.

4. The method of claim 1, wherein the refining comprises:
updating a group of pixels in the initial pathological element image assigned to a retinal pathological element of the set of retinal pathological elements to form the updated group of pixels for the retinal pathological element in the refined pathological element image by constraining an allowable area for the retinal pathological element based on the layer element image, wherein the updated group of pixels includes fewer pixels than the group of pixels.

5. The method of claim 1, wherein generating the layer element image comprises:
generating, via the first neural network, a multi-channel map using the OCT image, wherein the multi-channel map comprises a plurality of segmented images in which each segmented image of the plurality of segmented images identifies a corresponding retinal layer of interest.

6. The method of claim 5, wherein generating the layer element image further comprises:
converting the multi-channel map into an initial layer element image that identifies the set of retinal layer elements using the set of layer element indicators, wherein the set of layer element indicators assigns a different group of pixels in the initial layer element image to each retinal layer element of the set of retinal layer elements.

7. The method of claim 6, wherein the converting comprises:
applying piecewise logistic curve approximation to the multi-channel map to generate the initial layer element image.

8. The method of claim 6, wherein generating the layer element image further comprises:
applying smoothing to the initial layer element image to generate the layer element image.

9. The method of claim 8, wherein applying smoothing to the initial layer element image comprises applying Gaussian smoothing to the initial layer element image to generate the layer element image.

10. The method of claim 1, wherein a retinal layer element of the set of retinal layer elements is either a retinal layer or a boundary associated with the retinal layer.

11. The method of claim 10, wherein the retinal layer is selected from a group consisting of an internal limiting membrane (ILM) layer, an external limiting membrane (ELM) layer, an outer plexiform layer-Henle fiber layer (OPL-HFL), a retinal pigment epithelial (RPE) layer, a layer of RPE detachment, a Bruch's membrane (BM) layer, and an ellipsoid zone (EZ).

12. The method of claim 1, wherein the set of retinal pathological elements includes at least one of intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, or a disruption.

13. The method of claim 1, wherein each of the set of layer element indicators and the set of pathological element indicators includes at least one of a color indicator, a shape indicator, a pattern indicator, a shading indicator, a line, a curve, a marker, a label, a tag, or text.

14. The method of claim 1, wherein the first neural network comprises a first U-Net and the second neural network comprises a second U-Net.

15. The method of claim 1, wherein:
the first neural network is trained using a first training dataset comprising a first plurality of training OCT images and a plurality of training layer element images; and
the second neural network is trained using a second training dataset comprising a second plurality of training OCT images and a plurality of training pathological element images.

16. The method of claim 15, wherein at least a portion of the first plurality of training OCT images is included in the second plurality of training OCT images.

17. A method for performing retinal segmentation, the method comprising:
receiving an optical coherence tomography (OCT) image of a retina;
generating, via a neural network, a multi-channel map using the OCT image, the multi-channel map including a plurality of segmented images in which each segmented image of the plurality of segmented images identifies a corresponding retinal layer of interest;
generating a layer element image using the multi-channel map, identifying a set of retinal layer elements using a set of layer element indicators; and
refining an initial pathological element image using the layer element image to generate a refined pathological element image that visually identifies a set of retinal pathological elements using a set of pathological element indicators, wherein the refined pathological element image identifies at least one retinal pathological element in the set of retinal pathological elements more accurately than the initial pathological element image.

18. The method of claim 17, wherein generating the layer element image comprises:
converting the multi-channel map into an initial layer element image using piecewise logistic curve approximation.

19. The method of claim 18, wherein generating the layer element image further comprises:
applying smoothing to the initial layer element image to generate the layer element image that is then used to refine the initial pathological element image.

20. The method of claim 17, wherein refining the initial pathological element image comprises at least one of:
reassigning, based on the layer element image, a first portion of pixels in the initial pathological element image from one retinal pathological element to a different retinal pathological element in the refined pathological element image; or
reassigning, based on the layer element image, a second portion of pixels in the initial pathological element image to a background in the refined pathological element image.

21. A system for performing automated retinal segmentation, comprising:
a non-transitory memory; and
a data processor coupled with the non-transitory memory and configured to read instructions from the non-transitory memory to cause the system to perform operations comprising:
receiving an optical coherence tomography (OCT) image of a retina;
generating a layer element image using the OCT image and a first neural network, the layer element image identifying a set of retinal layer elements using a set of layer element indicators;
generating an initial pathological element image using the OCT image and a second neural network, the initial pathological element image visually identifying a set of retinal pathological elements using a set of pathological element indicators that assigns a different group of pixels to each retinal pathological element of the set of retinal pathological elements; and
refining the initial pathological element image using the layer element image to generate a refined pathological element image, the refined pathological element image visually identifying the set of retinal pathological elements using the set of pathological element indicators, the set of pathological element indicators assigning an updated group of pixels to at least one retinal pathological element of the set of retinal pathological elements.

22. A method for performing automated retinal segmentation, the method comprising:
receiving an image input for a retina of a subject;
generating layer element data using the image input and a first neural network, the layer element data identifying a set of retinal layer elements;
generating initial pathological element data using the image input and a second neural network, the initial pathological element data identifying a set of retinal pathological elements; and
refining the initial pathological element data using the layer element data to generate refined pathological element data, the refined pathological element data more accurately identifying the set of retinal pathological elements as compared to the initial pathological element data.

23. The method of claim 22, wherein the initial pathological element data comprises an initial pathological element image that visually identifies the set of retinal pathological elements using a set of pathological element indicators that assigns a different group of pixels in the initial pathological element image to each retinal pathological element of the set of retinal pathological elements.

24. The method of claim 23, wherein the refined pathological element data comprises a refined pathological element image that visually identifies the set of retinal pathological elements using the set of pathological element indicators, the set of pathological element indicators assigning an updated group of pixels to at least one retinal pathological element of the set of retinal pathological elements.

25. The method of claim 22, wherein the layer element data comprises a layer element image that identifies a set of retinal layer elements using a set of layer element indicators.

26. The method of claim 22, wherein the image input comprises an SD-OCT image.

27. The method of claim 22, wherein a retinal layer element of the set of retinal layer elements is either a retinal layer or a boundary associated with the retinal layer and wherein the retinal layer is selected from a group consisting of an internal limiting membrane (ILM) layer, an external limiting membrane (ELM) layer, an outer plexiform layer-Henle fiber layer (OPL-HFL), a retinal pigment epithelial (RPE) layer, a layer of RPE detachment, a Bruch's membrane (BM) layer, and an ellipsoid zone (EZ).

28. The method of claim 22, wherein the set of retinal pathological elements includes at least one of intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, or a disruption.
